(19) United States
(12) Patent Application Publication
(10) Pub. No.: US 2016/0109438 A1
(43) Pub. Date: Apr. 21, 2016

(54) PROTEASE-RESPONSIVE PEPTIDE BIOSENSORS AND METHODS FOR ANALYTE DETECTION

(71) Applicant: AGENCY FOR SCIENCE, TECHNOLOGY AND RESEARCH, Singapore (SG)

(72) Inventors: Saurabh Rajendra Nirantar, Singapore (SG); Farid John Ghadessy, Singapore (SG)

(21) Appl. No.: 14/895,696

(22) PCT Filed: Jun. 6, 2014
(86) PCT No.: PCT/SG2014/000263

§ 371 Date: Dec. 3, 2015

(30) Foreign Application Priority Data
Jun. 6, 2013 (SG) 201304395-5

(51) Int. Cl. G01N 33/53; G01N 33/68

(52) U.S. Cl. CPC G01N 33/5306; G01N 33/6845; G01N 2333/96433

(57) ABSTRACT

The present invention relates to peptide biosensors comprising (a) a protease recognition site; (b) an analyte binding site; and (c) a signaling moiety that can produce a detectable signal upon cleavage of the protease recognition site by a protease. Further encompassed are detection reagents that comprise said peptide biosensors in combination with a protease and methods for detecting the presence of an analyte molecule or screening of candidate compounds that modulate the binding of an analyte to a binding partner of the analyte.

### CROSS-REFERENCE TO RELATED APPLICATION

This application makes reference to and claims the benefit of priority of a Singapore patent application for “A Detection Method” filed on Jun. 6, 2013, and duly assigned application number 201304395-5. The content of said application filed on Jun. 6, 2013 is incorporated herein by reference for all purposes, including an incorporation of any element or part of the description, claims or drawings not contained herein and referred to in Rule 20.5(a) of the PCT, pursuant to Rule 4.18 of the PCT.

### FIELD OF THE INVENTION

The present invention relates to protease-sensitive peptide biosensors and methods for detecting the presence of an analyte molecule, in particular, recombinant peptide biosensors and methods for detecting the presence of an analyte molecule using analyte binding molecules.

### BACKGROUND OF THE INVENTION

It has been shown that more than 80% of proteins do not exhibit activity in the absence of complex formation, indicating the importance of protein-protein interactions in fundamental cellular processes. This observation has led to the relatively new endeavor of seeking antagonists of protein-protein interactions for therapeutic purposes. A large number of protein-protein interactions are mediated by specialized modular protein domains like PDZ, SH2, and SH3, which bind to cognate peptides in their respective interaction partners. In addition to the widespread usage of specialized peptide binding domains, it is estimated that in more than 50% of globular protein-protein interactions, the dominant contribution from one protein of the interacting pair can be reduced to a single peptide. Similarly, it has been shown that helical peptide segments form a major constituent of a number of protein-protein interactions which could be susceptible to small molecule inhibitors. Large numbers of peptide mimotopes, which can mimic one binding partner of a protein-protein interacting pair, have been discovered, typically using peptide phage display. Numerous databases and tools have been created to enable facile study of peptide-protein interactions. These facts point to the salience of peptide-protein interactions in the protein-protein interaction network. It would thus be very useful from a therapeutic perspective if novel methods could be developed to enable rapid and facile screening of drugs which can disrupt peptide-protein interactions.

Prevailing methods such as enzyme-linked immuno sorbent assay (ELISA), surface plasmon resonance (SPR) and fluorescence polarization (FP) can be adapted to study protein-protein interaction and inhibitor screening. However, none of these methods fulfill all the criteria desirable for drug screening, such as a homogenous set-up for facile high throughput screening, absence of washing and/or immobilization steps, robustness in the presence of a wide variety of auto-fluorescent small molecule drugs, presence of serum, cell lysates and other complex fluids, and a turn-on instead of a turn-off signal in response to an inhibitor. For example, ELISA and SPR are time and labor intensive, whilst fluorescence polarization is a turn-off method which can suffer interference from auto-fluorescent drugs or small metabolites.

Other homogenous methods such as the protein fragment complementation assay (PCA) typically give a turn-off signal in response to interaction inhibitors and require the fusion of split protein domains to the interacting proteins. Therefore, in order to meet the requirements of high throughput screening and accelerate the detection of antagonists of protein-protein interaction with high sensitivity and minimal process time, provision of a homogenous screening system with high specificity and robustness is highly desirable.

Generally, one-step homogenous biosensors also have the potential to significantly simplify and expedite analyte detection procedures as tedious washing steps or secondary detection reagents (like HRP labeled antibodies) that are the norm with current procedures such as ELISA are not required. Accordingly, such analyte responsive biosensors that can greatly facilitate many laboratory procedures are also much sought after in the field. While such biosensors for analyte detection are for example described in the international patent publication WO 2012/128722, screening applications for inhibitors of protein-protein interactions are not mentioned and these biosensors would also give a turn-off signal in response to an inhibitor.

The inventors of the present invention have developed such a method by extrapolating the principles of the nuclease protection assay into a protein based system that meets the above-described needs and can be used for analyte detection as well as screening of drug candidates that can interfere with peptide/protein-protein interactions. The newly developed method is demonstrated herein using both fluorescence and enzyme-coupled readout formats and has been validated in a small-molecule (fragment) screen for inhibitors of the p53-Mdm2 interaction, where it demonstrated high sensitivity and specificity compared with other methods.

### SUMMARY OF THE INVENTION

In a first aspect, the invention relates to a peptide biosensor for the detection of an analyte, wherein the peptide biosensor comprises (a) a protease recognition site; (b) an analyte binding site; and (c) a signaling moiety that can produce a detectable signal upon cleavage of the protease recognition site by a protease.

In certain embodiments of the peptide biosensor, the protease recognition site and the analyte binding site are positioned relative to each other such that binding of the analyte to the analyte binding site reduces or prevents binding of the protease to the protease recognition site. This may, for example, mean that the protease recognition site and the analyte binding site are directly adjacent to each other or separated only by a linker of up to 20 amino acids.

In another aspect, the invention is directed to a detection reagent for the detection of an analyte, wherein the detection reagent comprises (a) the peptide biosensor as described herein and (b) a protease capable of binding to and cleaving the protease recognition site. In certain embodiments, the protease capable of binding to and cleaving the protease recognition site is coupled to an analyte binding molecule and the analyte can simultaneously be bound by the analyte binding site of the biosensor and the analyte binding molecule and the protease recognition site and the protease are selected such that cleavage of the protease recognition site by the protease is detectably increased if both, the peptide biosensor and the protease, are bound to the analyte.

In still another aspect, the invention also relates to a method of detecting the presence and/or amount of an analyte in a sample, the method comprising: (i) contacting the detection reagent according to the invention with a sample suspected of containing the analyte under conditions that allow binding of the analyte by the detection reagent; and (ii) detecting the presence and/or amount of the analyte in said sample by measuring the signal of the signaling moiety.

A still further aspect of the invention encompasses a method of screening for compounds that modulate the binding of an analyte to a binding partner of the analyte, the method comprising (i) contacting the detection reagent according to the invention with the analyte and a candidate compound under conditions that allow binding of the analyte by the detection reagent, wherein the analyte binding partner is the analyte binding site or the analyte binding molecule; and (ii) measuring the signal of the signaling moiety, wherein a change in the signal compared to a reference not containing the candidate compound indicates modulation of the binding of the analyte and the analyte binding site or analyte binding molecule by the candidate compound.

In still another aspect, the invention also relates to a kit for detecting the presence and/or amount of an analyte in a sample, the kit comprising one or more detection reagents as described herein.

### BRIEF DESCRIPTION OF THE DRAWINGS

In the following description, various embodiments of the invention are described with reference to the following drawings, in which:

FIG. 1 shows (A) a schematic depiction of the biosensor concept. An enzyme (¾th circle) is fused to its own inhibitor (triangle) via a peptide linker containing a protease site. In this drawing the enzyme Tem1 lactamase is inhibited due to the close proximity of its inhibitor BLIP, but other enzyme-inhibitor pairs may also be used. Upon cleavage at the protease site by the protease (scissors), the inhibitor can diffuse away, leading to increased enzyme activity. (B) Example of an enterokinase protease sensor based on the scheme depicted in (A) showing the sensor response upon various amounts of enterokinase. Increasing amounts of enterokinase lead to increased amounts of sensor cleavage, generating a proportionally greater signal.

FIG. 2A shows a schematic drawing of the biosensor concept of the present invention based on the concept depicted in FIG. 1A, but with a ligand binding site (light line) adjacent to the protease recognition and cleavage site (dark line). Binding of the ligand to its cognate peptide causes steric hindrance to the protease seeking to access its recognition site. This keeps signaling enzyme activity low. Upon the addition of a drug or other antagonist (small triangle) of the peptide-ligand interaction, ligand is displaced from its recognition peptide, enabling protease to access its cleavage site. Cleavage of the linker causes enzyme activity to increase due to inhibitor dissociation. Thus the presence of an antagonist of peptide-protein interaction leads to increased enzyme activity. (B) Substrate turnover rate after treatment of Mdm2-enterokinase sensor with various concentrations of Nutlin, wildtype p53 peptide and mutant p53 peptide. (C) Western blot analysis results of different samples in Mdm2-enterokinase sensor assay, lane 1: sensor with enterokinase only, lane 2: sensor with enterokinase, Mdm2 and wildtype p53 peptide, lane 3: sensor with enterokinase, Mdm2 and mutant p53 peptide, lane 4: sensor with enterokinase, Mdm2, lane 5: sensor with enterokinase, Mdm2 and Nutlin.

FIG. 3 shows reactions comprising HA protease exclusion sensors upon addition of free peptides, HA-antibody and protease. (A) Reactions comprising the enterokinase-HA protease exclusion sensor, enterokinase and HA antibody were set up with varying amounts of free HA peptide as indicated. (B) Thrombin and HA antibody were set up with varying amounts of free HA or non-specific peptide as indicated. The rate of substrate turnover increases with increasing amount of free HA peptide but not non-specific peptide; consistent with the protease exclusion concept. The decrease seen with 4000 nM HA peptide might be due to the effect of the higher amounts of DMSO in the reaction as increasing amounts of DMSO dissolved peptide are added to the reaction. (C) HA-enterokinase sensor was treated with the indicated amounts of F-7 HA antibody (grey) in the presence of 1.2 nM enterokinase. As predicted, increasing amounts of HA antibody, but not non-specific whole mouse IgG (black data point), inhibits enterokinase cleavage mediated Tem1 activation, as measured by substrate turnover at OD492. (D) HA-enterokinase sensor, was treated with various concentrations of enterokinase in the absence of HA antibody (grey data points). An excess of TEV protease was added as a control (black data point). (E) HA enterokinase sensor treated with various amounts of free HA peptide, in the presence of 1.2 nM enterokinase and F-7 HA antibody.

FIG. 4 shows various tests of the Mdm2-protease exclusion sensor based on a Mdm2-p53 peptide interaction. (A) Primary screening result of all 352 fragments from the Zenobia fragment library. For each plate, drugs from each 8 well column were transposed into a row on the assay plate and each row has a negative (dark grey) and a positive control (light grey) flanking the drugs. For each row, the rate of substrate turnover for the negative control was normalized to 1 and the results are as shown. The dotted line indicates the cut-off threshold. (B-D) 15 positive fragments, a non-reactive fragment (negative control) from the screen and Nutlin (positive control) were tested in a competitive fluorescence polarization assay with a FAM labeled Mdm2 binding peptide (12-1) and purified Mdm2 N-terminus, the highest concentration of the drugs is 1 mM while that of Nutlin is 50 μM. (E) Determination of p53-Mdm2 interaction and modulation by small molecules inhibitors screened using a Mdm2-p53 protease exclusion sensor screen using the sensors system shown in FIG. 2, using in vitro pull-down and qPCR. Assay measures the amount of DNA (in complex with p53) pulled-down on to beads coated with Mdm2 N-terminal domain protein. Percentage binding denotes the amount of DNA pulled down in presence of indicated small molecules (10 μM for Nutlin, 1 mM for fragments) compared to DMSO control. Blank reaction indicates DNA pulled-down in absence of Mdm2. Values represent mean±SD (n=2).

FIG. 5 shows a test of the enterokinase eiF4E protease exclusion sensor. (A) The response of the sensor treated with eiF4E protein and enterokinase, along with various concentrations of free eiF4E peptide (closed circle). The rate of substrate turnover is plotted on the Y axis. The rate of turnover seen in the absence of eiF4E protein is shown as the data point in the Y axis (black triangle). As control, a non-specific wild type p53 peptide (square) at the highest concentration used for the eiF4E peptide was also assayed. (B) Dose response of eiF4E-enterokinase sensor to the 5 best hits (Fragments B, D, G, I and K) obtained from the Mdm2-enterokinase sensor secondary screening, demonstrating their specificity for the Mdm2-p53 interaction.

FIGS. 6 and 7 show the concept and validation of a protease exclusion sensor using a synthetic internally quenched peptide. (6A) In this sensor the peptide has a fluorophore (star) at the N-terminus, followed by a protease site, which is cleaved by enterokinase, a single amino acid glycine linker, a quencher (circle) attached to the N-terminal site of a p53 based peptide sequence which binds the Mdm2 N-terminus (oval). As the fluorophore s placed close to a quencher, the emission intensity will be low. The bulky Mdm2 hinders enterokinase access to its protease site due to steric clashes with Mdm2 N terminus bound immediately adjacent to the enterokinase site, preventing the rapid increase in fluorescence, which follows enterokinase cleavage. Addition of a small molecule drug (triangle) or a peptide which is a competitive inhibitor of the Mdm2-p53 peptide interaction sequesters the Mdm2 N terminus, thereby restoring enterokinase access to its protease site, causing increased fluorescence. (6B) The peptide configuration shown in FIG. 6A shows a strong response to Nutlin (light curve with circles) and the WT p53 peptide (squares) but not the non-binding mutant peptide (triangles). A certain amount of background cleavage was seen to occur. The signal to noise ratio was ˜5-6× with low micromolar concentrations of Nutlin/WT peptide being detectable. The rate of fluorescence increase was plotted as the difference between timepoints 3 and 1. FIG. 6C depicts 15 compounds that successfully inhibited Mdm5-p53 interaction and were identified in a screening method according to the invention.

FIG. 7 shows a similar concept to 6A, except that the quencher was located C-terminal of the p53 based peptide.

FIG. 8 shows the concept and tests of the peptide biosensor using analyte enhanced protease signaling. (A) Scheme of a peptide biosensor using analyte enhanced protease signaling modified such that the protease site was sub-optimal. Hence, not much cleavage occurs at the site, leading to low signal generation. The protease and its sensor are fused to moieties that recognize an analyte. When the analyte is introduced, these moieties bind, thus bringing the protease and its sensor into close proximity. Here, the analyte is an HA-antibody, the protease is linked to HA epitope while the sensor is linked to protein L, which binds the variable domain of antibodies. Binding of both protease and sensor to the same HA antibody leads to a much greater effective concentration of both sensor and protease, enabling the protease to cleave the linker despite the suboptimality of the site, leading to signal generation. (B) The experiment described in FIG. 8A was conducted with various amounts of HA antibody. OD492 measurements were taken to monitor the resulting substrate turnover. (C) An analyte enhanced sensor system was set-up in which both protease and sensor were tagged with HA epitope. Addition of various HA antibodies (F7, C5 and Rab-HA) to this system leads to dose dependent signal generation, but not with non-specific antibodies (2A9 and Rab-P) nor in the absence of antibody. (D) As in C, but using myc epitope tag instead of HA tag. Myc antibodies (9B11 and 9E10) give dose dependent positive signals whereas non-specific antibodies (DO12, 2A9 and Rab-P) do not, nor does the absence of antibody.

FIG. 9 (A) shows a schematic drawing of the sensor concept as depicted in FIG. 8A, except that protease and sensor are linked to protein A that binds to the heavy chains of an antibody. (B) Dose response of various IgGs as indicated. The Y-axis referees to rate of increase in OD492 per minute, which is a measure of Tem1 signaling enzyme activity. Mouse IgG2a antibodies (2A9 and DO1), which bind protein A with high affinity, show a string response, while mouse IgG1 antibody (DO12) shows a weaker response.

FIG. 10 shows the concept of the protease exclusion sensor using two Fab antibody fragments. Fab fragments of antibodies binding on two different sites on analyte are added, along with protein L fused protease as well as protein L fused sub-optimal sensor. The sensor and protease bind to the analyte bound Fab antibodies, thereby coming in close proximity of each other. This increases the rate of turnover, leading to signal generation.

FIG. 11 shows schematic drawing of a substrate(chip)-based protease exclusion sensor. (A) A capture antibody and enzymes coupled to a peptide-linker with suboptimal protease site are bound on a chip matrix in close proximity to each other. Immobilized (enzyme) substrate is located distant-to the enzyme such that no enzymatic reaction between fixed substrate and enzyme is possible. A free detection protease-conjugated antibody and an analyte are added. (B) The analyte binds both antibodies, leading to the cleavage of enzyme and peptide linker by the protease. (C) The freed enzyme is released from the peptide tether (D) The enzyme diffuses to substrate leading to a detectable reaction.

FIG. 12 shows exemplary linker peptide sequences used in the sensors of the invention.

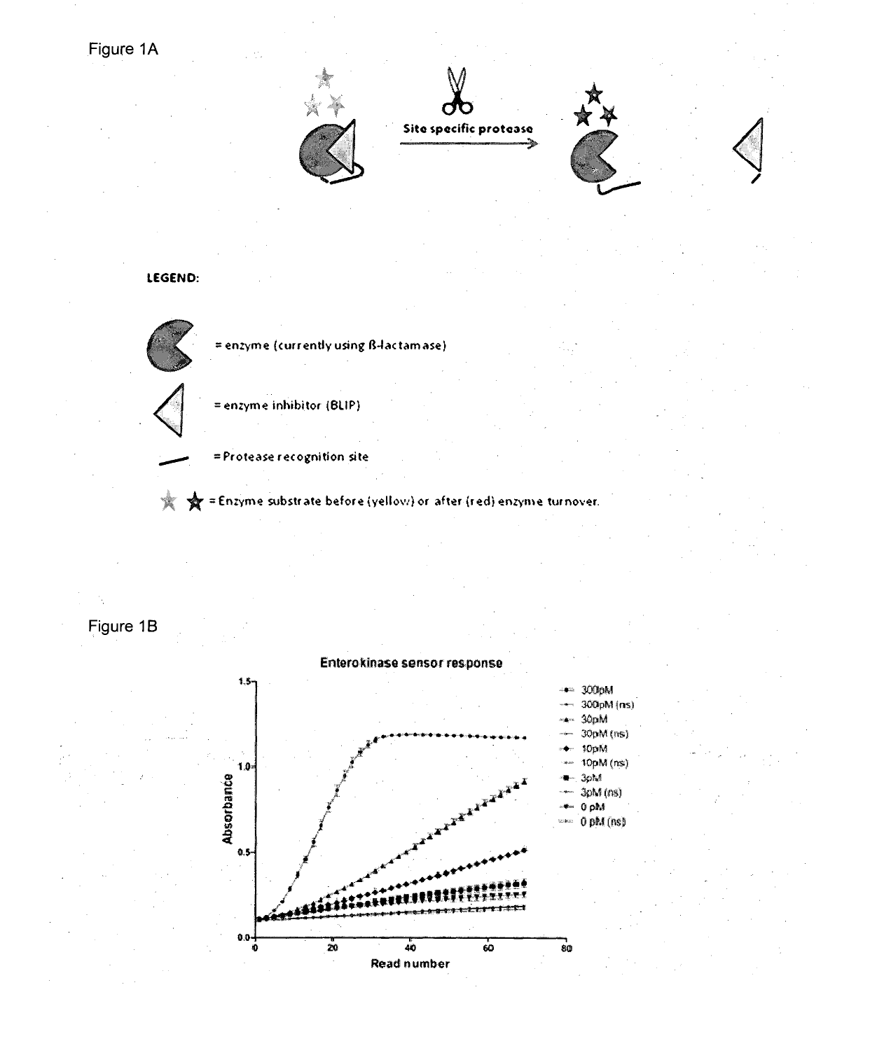

### DETAILED DESCRIPTION OF THE INVENTION

The following detailed description refers to, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

### Peptide Biosensors

As already disclosed above, the invention features, in a first aspect, a peptide biosensor for the detection of an analyte, the peptide biosensor including (a) a protease recognition site; (b) an analyte binding site; and (c) a signaling moiety that can produce a detectable signal upon cleavage of the protease recognition site by a protease.

The peptide biosensor is “protease-responsive” in that it is susceptible to protease-mediated cleavage and upon cleavage generates a detectable signal that allows distinguishing between the non-cleaved and cleaved form of the peptide biosensor.

The term “peptide biosensor”, as used herein, relates to a peptide-based molecule that allows detection of the presence and/or amount, preferably both, of an analyte. The term “peptide”, as used in this context, i.e. in connection with the peptide biosensor, relates to a polymer of amino acids that are linked by peptide bonds, with said peptide having a length that is sufficient to provide for a protease recognition site and an analyte binding site. Typically, the peptide biosensors comprise at least 10-15, preferably at least 25 amino acids. Depending on the length of the peptide, the peptide may be considered a polypeptide, with such polypeptides typically having a length of 50 or more amino acids. The term “(poly)peptide”, as used herein, is intended to include both peptides and polypeptides. While the term “polypeptide”, as used herein, refers to a single (poly)amino acid chain, the term “protein”, as used herein, relates to macromolecules that consist of one (in which case the term is interchangeable with “polypeptide”) or more polypeptide chains. In various embodiments, the peptide biosensors are 10-500 amino acids in lengths. In other embodiments they are 15-100 amino acids in length. In still other embodiments, they are 25-50 amino acids in length.

The term “amino acid” refers to naturally occurring and artificially produced amino acids, and also amino acid analogs and amino acid mimetics that function in a similar manner to the naturally occurring amino acids. Amino acid analogs refer to compounds that have the same basic chemical structure as the naturally occurring amino acids. It is preferred that in the peptides of the present invention, the 20 naturally occurring amino acids glycine, alanine, valine, leucine, isoleucine, phenylalanine, proline, cysteine, methionine, tyrosine, tryptophan, glutamine, asparagine, serine, threonine, glutamic acid, aspartic acid, histidine, lysine and arginine are used.

In various embodiments, the peptide biosensor may be a fusion protein. In the context of various embodiments, the term “fusion” or “fusion protein” refers to two or more (poly)peptides, including protein fragments, covalently linked via peptide bonds and the respective peptide backbones. Typically, a fusion protein is an artificial protein or polypeptide derived from fusing the two or more proteins or fragments thereof. In some examples, the proteins and fragments thereof may originate from different sources, e.g. may be heterologous. In various embodiments the fusion proteins result from fusing peptides, polypeptides, proteins or fragments thereof such that they are not identical to a peptide, polypeptide or protein that occurs in nature, i.e. respective amino acid sequence stretches are artificially combined in a single protein/polypeptide.

The analyte is preferably a bio(macro)molecule, more preferably a (poly)peptide or protein or a nucleic acid, most preferably a polypeptide or protein.

The protease recognition site comprises an amino acid sequence that is recognized and bound by a protease. Once bound, the protease cleaves the peptide biosensor. The actual cleavage site may form part of the protease recognition site or may lie N-terminal or C-terminal to the recognition site. Typically, the protease recognition site comprises the cleavage site in that the peptide bond between two amino acids within the protease recognition site or the peptide bond directly C-terminal or N-terminal to the recognition site is cleaved, usually hydrolyzed. In various preferred embodiments, the proteases used in accordance with the present invention are endopeptidases, i.e. enzymes that cleave peptide bonds of non-terminal amino acids. The protease used will be selected depending on the protease recognition site, which will in turn be selected based on the desired properties of the peptide biosensor. Suitable protease:binding site pairs can be readily selected by those skilled in the art. Exemplary proteases include, without limitation, enterokinase (enteropeptidase), thrombin, TEV protease (Tobacco Etch Virus nuclear inclusion A endopeptidase), SpIB protease, HRV3C protease, TVMV protease, chymotrypsin and the like.

In one embodiment, the protease recognition site is DDDDR (SEQ ID NO:1) and the protease is enterokinase.

The analyte binding site is an amino acid sequence in the peptide biosensor that is recognized and bound by an analyte of choice. The recognition and binding may be “specific”, i.e. the analyte may preferentially bind to the analyte binding site compared to other random amino acid sequences. Such preferential binding may mean that the binding affinity is at least 10 fold, preferably at least 100 fold higher compared to the binding affinity of any one of the binding partners for other non-target peptides, polypeptides and proteins that show no substantial sequence homology with the respective binding partner The binding is preferably non-covalent binding, for example, without limitation, by hydrogen bonding, van der Waals forces, π-π stacking and/or electrostatic (ionic) interactions.

The analyte binding site may be of any desirable length and may be designed based on the selected analyte. Typically, the length varies between about 6 and about 50 amino acids, but may be longer in case protein-protein interactions are involved, i.e. the analyte and the analyte binding site are proteins or derived from proteins. The analyte binding site may be derived from a longer protein sequence and comprise only those parts of the protein that are needed for analyte binding.

In various embodiments, the analyte binding site is a first (poly)peptide capable of binding the analyte, preferably capable of specifically binding the analyte. The first (poly)peptide may be a naturally occurring sequence, i.e. be identical to a part or the complete sequence of a peptide or protein that occurs in nature, but may alternatively be artificially designed, for example by mutation of a known binding site. Such artificial binding sites may be designed such that binding affinity for a given analyte is increased or decreased or a given specificity is altered.

In various embodiments, the first (poly)peptide comprises or consists of an epitope.

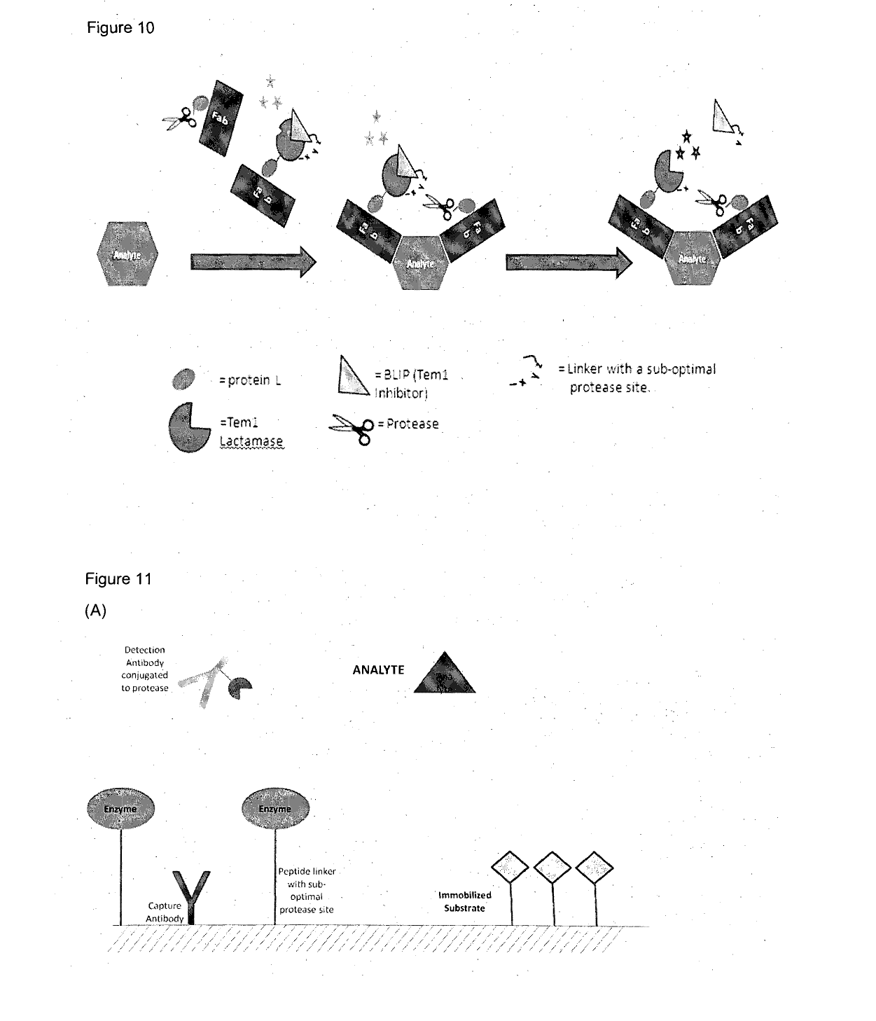

The binding of the analyte binding site or first (poly)peptide to the analyte may be direct or indirect. In case of direct binding, the analyte binding site or first (poly)peptide directly contact and bind the analyte. In case of indirect binding, the analyte binding site or first (poly)peptide bind to another molecule which is a binding partner of the analyte and in turn binds the analyte. The binding partner of the analyte may be bound by the analyte binding site or the first (poly)peptide by covalent or non-covalent, preferably non-covalent, interactions and may in turn bind the analyte by covalent or non-covalent, preferably non-covalent, interactions. Exemplary binding partners include, but are not limited to antibodies, antibody-like and antibody-derived molecules and fragments thereof. These may for example be attached to the analyte binding site/first (poly)peptide by non-covalent complex formation via a part different from the antigen-binding region. Accordingly, the analyte binding site/first (poly)peptide capable of binding the analyte may be a peptide or protein (fragment) that binds another scaffold peptide or protein fragment which then in turn binds the analyte. The first (poly)peptide may thus for example be protein L or protein A, the scaffold may be an antibody or antibody fragment, like the Fab antibody fragment, with said antibody having antigen-binding regions specific for the analyte. Such an embodiment is depicted in FIG. 10.

Exemplary analyte binding sites/first (poly)peptides include, without limitation, antibodies, antibody fragments, peptide aptamers, peptide antigens, or peptide antigen fragments as well as protein fragments, in particular those protein fragments that comprise the binding site for another protein.

As used herein, the term “antibody” may be used in the broadest sense and covers polyclonal antibodies, monoclonal antibodies, multispecific antibodies, single domain antibodies, and phage antibodies. Antibodies may refer to fragments of antibodies. The term “antibody fragment” means a portion of the full length antibody, generally the antigen binding or variable region thereof. For example, an antibody fragment may include single chain antibodies (scFv) or binding fragment (Fab). Antibodies may be interchangeably referred to as immunoglobulin. Varieties of antibodies may be, for example, IgA, IgD, IgE, IgG and IgM. Examples of antibodies may be but not limited to anti-HIV pI7 epitope, p53 (DO-1) monoclonal antibody, and anti c-myc antibody.

In some examples, the analyte binding site may comprise naturally occurring ligands or interacting partners. For example, mdm2 may be used as the analyte binding site for the detection of p53 or vice versa. It is however preferred in case the interacting partner are proteins that the analyte binding site only comprises the binding site of the respective protein for its interacting partner. Accordingly, in one embodiments of the invention the analyte binding site is the mdm2-binding site of p53 and the analyte is mdm2.

The “peptide aptamer” may be a combinatorial protein reagent that binds to target proteins with a high specificity and a strong affinity. For example, the peptide aptamer may inhibit the function of a protein in vivo.

The term “antigen” generally refers to a molecule capable of being bound by an antibody. For example, an antigen may be but is not limited to pathogen derived proteins/molecules, molecules of medical interest such as insulin, hcG, etc. In one embodiment, the antigen or antigen fragment may comprise or consist of an antigenic determinant or epitope.

In specific embodiments, the analyte binding site may be the influenza hemagglutinin epitope (HA epitope), for example having the amino acid sequence YPYDVPDYA (SEQ. ID NO:2), a mdm2-binding peptide based on p53, for example having the amino acid sequence TSFAEYWNLLSP (SEQ ID NO:3), and the like.

The signaling moiety is capable of producing a detectable signal upon cleavage of the protease recognition site. The signaling moiety comprised in the” peptide biosensor may also comprise or consist of one or more (poly)peptide(s).

As used with reference to the signaling moiety herein, the term “produce” may interchangeably be referred to generate, send, give off, give or emit. The term “detectable signal” refers to a signal that can be detected or measured directly or indirectly. For example, the detectable signal may be detectable or measurable by physical, spectroscopic, photochemical, biochemical, immunochemical or chemical means. The detectable signal may be produced directly or indirectly by reaction or interaction with a suitable conjugate, for example, a substrate. The detectable signal may be an “indicator molecule”.

The signaling moiety is an indicator for the state of the peptide in that the signal produced is changed based on whether the signaling moiety is present in a non-cleaved peptide biosensor or a cleaved peptide biosensor. Accordingly, the term “upon cleavage of the protease recognition site”, as used herein in connection with the signaling moiety, means that the signal produced by the signal moiety detectably changes once the protease recognition site has been cleaved by the protease, i.e. the signal produced by the signaling moiety detectably differs between the cleaved and the non-cleaved state of the sensor. This detectable change may, for example, be signal generation of the cleaved biosensor (compared to a non-cleaved biosensor not producing such a signal) or termination of a signal produced by the non-cleaved biosensor after cleavage. It is understood that the signal may be continuously produced in the cleaved or the non-cleaved state or both, with the latter embodiment additionally requiring that the signal detectably differs between both states.

The signaling moiety may comprise a signal generating moiety, such as an enzyme, fluorophore or chromophore and optionally a modulator thereof.

In the context of various embodiments, the term “modulates” means change. For example, the signal may be modulated in that it is increased or decreased. Accordingly, the modulator may activate or promote signal generation by the signal generating moiety upon binding or, alternative, prevent or inhibit signal generation by the signal generating moiety upon binding. Accordingly, the modulator may be an activator or an inhibitor.

In embodiments where the signaling moiety comprises or consists of a signal generating moiety and a modulator, the modulator binds to the signal generating moiety and, when bound to the signal generating moiety, modulate's the signal generation by the signal generating moiety. The interaction between signal generating moiety and modulator is preferably sensitive for the cleavage of the protease recognition site such that cleavage thereof by a protease changes, i.e. typically interferes with, the binding of the modulator to the signal generating moiety.

For example, but without limitation, the signal generating moiety may be a (poly)peptide coupled to a substance that can produce the detectable signal. Said substance may for example be a fluorophore or chromophore, but is not limited thereto.

In other embodiments, the signal generating moiety is an enzyme or has enzymatic activity. The enzymatic activity may be conferred by an enzyme fragment that retains all or part of the original enzyme's activity.

The enzyme may be a catalytic peptide and/or may be capable of providing a convenient read-out. For example, the enzyme may be selected from the group consisting of lactases, catalases, amylases, beta-lactamases, cephalosporinases, penicillinases, cephalosporinases, carbenicilliniases, beta-galactosidases and alkaline phosphatases, luciferase and others.

In one embodiment, the enzyme may be beta-lactamase or homologs, fragments and variants thereof, wherein the homology, fragments and variants at least partially retain enzymatic activity.

Generally, as used herein, the term “beta lactamase” includes multiple beta lactamases, for example, any of Class A beta lactamases, Class B beta lactamases, Class C beta lactamases, and/or Class D beta lactamases. In one embodiment, the beta lactamase is a Class A beta lactamase, such as, for example, TEM1.

In one specific embodiment, the enzyme is the beta lactamase TEM1 or variants thereof. “Variant”, as used herein, refers to a protein that differs from a consensus sequence or the accepted wildtype sequence by at least one amino acid variation. The variant may be but is not limited to a natural variant, or a M69L variant, or a E104K variant. The beta lactamase TEM1 may comprise the amino acid sequence set forth in SEQ ID NO:4 (UniProtKB accession number Q5QJI7). In one specific embodiment, the beta lactamase TEM1 may comprise a homolog or fragment or variant of the amino acid sequence set forth in SEQ ID NO: 4.

“Homologs”, as used herein, refer to two proteins that have similar amino acid sequence. Homologs include orthologs, or paralogs. “Fragments”, as used herein, refer to a portion of amino acid sequence, that is, a polypeptide comprising fewer than all of the amino acid residues of the protein.

In still further embodiments, the signal generating moiety may be a fluorescent protein or peptide.

In case the signal generating moiety comprises a fluorophore, the fluorophore may be positioned C- or N-terminally of the protease recognition site. In such embodiments, a modulator of the fluorophore, preferably a quencher is also present with said quencher being positioned at the other side of the protease recognition site such that the protease recognition site lies between the fluorophore and the quencher.

Specifically, the fluorophore may be positioned N-terminally to the protease recognition site, more preferably at the N-terminus of the protease responsive fusion protein/peptide. The quencher may either be positioned directly adjacent the protease recognition site, i.e. between the protease recognition site and the first (poly)peptide, or positioned C-terminally of the peptide capable of binding the analyte, for example at the C-terminus of the protease responsive fusion protein/peptide, provided the protease recognition site is located between the fluorophore and the quencher.

In various embodiments, the fluorophore may be 5-((2-Aminoethyl)amino)naphthalene-1-sulfonic acid (EDANS), for example conjugated as a side chain to a glutamic acid residue. EDANS fluorescence may be measured by a fluorescence spectrometer at 490 nm using an excitation wavelength of about 335 nm. The quencher may be 4-(dimethylaminoazo)benzene-4-carboxylic acid (Dabcyl). Dabcyl may for example be conjugated to the side chain of a lysine residue.

Other suitable fluorophores include, without limitation, fluorescein, isothiocyanate, coumarin, cyanine and rhodamine.

Other suitable quenchers that may be selected based on the fluorophore selection include, but are not limited to, dark quencher, dimethylaminoazosulfonic acid, black hole quenchers, and Qxl quencher.

In various embodiments, the modulator comprises or consists of a (poly)peptide. It is preferred that both, the modulator and the signal generating moiety comprise or consist of a (poly)peptide.

The modulator may be a (poly)peptide coupled to a substance that can modulate the detectable signal produced by the signal generating moiety. Said modulator substance may for example be a quencher for a detectable signal producing substance that is fluorophore or chromophore.

In other embodiments where the signal generating moiety is an enzyme or has enzymatic activity, the modulator can be an activator or inhibitor of said enzyme or said enzymatic activity:

For illustrative purposes, an example of an enzyme activator may be the fragment of beta-galactosidase and variants thereof, which activates the [Omega] fragment. Other enzyme activators may work in a similar manner. For example, an enhancer may be an enzyme enhancer, which can bind to a non-active site and cause a conformation change which enhances enzyme function.

When the signaling moiety consists of a signal generating moiety and a modulator, it is preferred that the protease recognition site is located between the signal generating moiety and the modulator. This ensures that in case the protease recognition site is cleaved by the protease, signal generating moiety and modulator are no longer present in the same molecule, but are subject to diffusion processes making the influence of the modulator on the signal generating moiety less pronounced.

In specific embodiments of this aspect of the invention, the signal generating moiety comprises or consists of an enzyme and the modulator comprises or consists of an inhibitor of said enzyme. The linkage of the enzyme and the enzyme inhibitor may be designed such that upon cleavage of the protease recognition site either the enzyme or the enzyme inhibitor is released from the protease responsive fusion protein. The inhibitor may be a competitive inhibitor or an allosteric inhibitor. Binding of the inhibitor to the enzyme is reversible. For example, the inhibitor may be but is not limited to a protein inhibitor such as alpha 1-antitrypsin, C1-esterase inhibitor, antithrombin, alpha 1-antichymotrypsin, plasminogen activator inhibitor-1, neuroserpin, or a beta-lactamase inhibitor protein (BLIP), or inhibitor peptides/proteins discovered by screening.

In embodiments where the signal generating moiety is or comprises a beta-lactamase, the inhibitor may be beta lactamase inhibitor protein (BLIP) or BLIP-I or BLIP-II or fragments, homologs and variants thereof that retain at least partially the binding activity for beta lactamase. In one specific embodiment, the modulator is a beta lactamase inhibitor protein (BLIP) or variants thereof. The terms “variant”, “homologs” and “fragments” are as defined above. The BLIP may have the amino acid sequence set forth in SEQ ID NO:5 (UniProtKB accession number Q18BP3). In one specific embodiment, the BLIP may comprise a homolog or fragment or variant of the amino acid sequence set forth in SEQ ID NO: 5, in particular it may be the D49A mutant of the protein set forth in SEQ ID NO:5.

In the above embodiments, the enzyme is generally selected such that it can, in the presence of a suitable substrate, produce a detectable signal due to its catalytic activity. Adding an enzyme substrate to the incubation reaction will thus result in a detectable signal, provided that the protease recognition site has been recognized and cleaved by the protease so that either the enzyme inhibitor or the enzyme has been released from the protease responsive fusion protein and their interaction is reduced.

In other embodiments, the signal generating moiety comprises or consists of a fluorophore or, chromophore and the modulator comprises or consists of a quencher of said fluorophore or chromophore. As already described above, in such embodiments the signal generating moiety may comprise a (poly)peptide that is coupled to the fluorophore or chromophore, either via the terminus or via a side chain of an amino acid residue.

Some exemplary signal generating moiety:modulator pairs that may be used in accordance with various aspects of the invention include, but are not limited to, a β-lactamase enzyme, such as TEM1, and an inhibitor of TEM1, such as BLIP (beta-lactamase-inhibitor protein) or a fluorophore, such as EDANS, and a quencher thereof, such as 4-(dimethylaminoazo)benzene-4-carboxylic acid (Dabcyl).

In all embodiments, where a fluorophore is used in combination with a quencher, the quencher may be replaced by another fluorophore. In such a case, the detection makes use of the so-called fluorescence resonance energy transfer (FRET) technique, where the one fluorescent moiety, when in close proximity to the other, will absorb photons emitted by the other fluorophore via Fluorescence Resonance Energy Transfer (FRET) and re-emit a longer wavelength photon. In certain embodiments, the first fluorophore may be green fluorescent protein (GFP) and the other fluorophore may be red fluorescent protein (RFP). In case of using GFP and RFP, for example, the GFP emission photons, are not seen when RFP is in close proximity; as RFP is inhibiting GFP emission so that only RFP emission is detected. When RFP is moved away from GFP more of the GFP emission photons will be detected and a GFP signal is detectable. In this way, a FRET acceptor such as RFP may also be considered an inhibitor of a FRET donor (such as GFP). However, in preferred embodiments of the invention the detection principle is not based on FRET, but rather on the fluorophore:quencher or enzyme:inhibitor interaction.

As described above, the peptide biosensor may be a fusion protein, said fusion protein comprising in N- to C-terminal orientation a structure selected from the group consisting of structures (I)-(VIII):
B-P-C-A (I);
C-P-B-A (II);
C-A-P-B (III);
C-P-A-B (IV);
B-A-P-C (V);
B-P-A-C (VI);
A-B-P-C (VII); or
A-C-P-B (VIII)
wherein
A represents a (poly)peptide capable of binding the analyte, preferably capable of specifically binding the analyte;
B represents a signal generating moiety that can generate a detectable signal;
C represents a modulator that is capable of binding to and modulating the signal generation by B;
P represents the protease recognition site; and
“-” represents a covalent bond or a peptide linker comprising or consisting of one or more amino acids.

A may be defined as the first (poly)peptide above.

B may be selected from the signal generating moieties described above. Similarly, C may be selected from the modulators described above. The length of the linker between B and C is selected such that it allows the interaction of B and C. In other words, the part of the molecule linking B and C, including P and optionally also A, has to be long enough to allow this interaction. This of course similarly applies to the signaling moiety in cases where it comprises a signal generating moiety and a modulator.

The protease recognition site P may also be defined as the protease recognition sites that have been described above.

The term “structure”, as used in this connection, refers to a chemical structure, more specifically the architecture of a peptide or polypeptide with respect to the location and order of the separate functional domains.

As used herein, the term “domain” with reference to a peptide, polypeptide or protein may refer to a functional unit of the peptide biosensor. In certain embodiments, the term may cover an independently folding peptide structure that may naturally be part of a larger protein. For example, a domain in the sense of the present invention may include one or more amino acid stretches that have a secondary, optionally a tertiary and optionally a quaternary structure and fold independently from other parts of the protein that may not be present in the isolated domain.

Generally, in the peptide biosensors having any one of structures (I)-(VIII), the modulation of the signal generation by B by the modulator C detectably varies between the cleaved and non-cleaved state of the peptide biosensor. This is due to the fact that in the cleaved state either B or C is released and thus no longer held in close proximity/bound to the remaining entity with the result that the interaction is weakened and the signal is less influenced by the modulator.

In various embodiments of the invention, the protease recognition site and the analyte are positioned relative to each other, for example adjacent to each other, such that binding of the analyte to the analyte binding site reduces or prevents binding of the protease to the protease recognition site. This reduction or prevention of protease binding to the protease recognition site is effected by designing the arrangement of the separate units, i.e. the protease recognition site and the and the analyte binding site/first (poly)peptide capable of binding the analyte, that the accessibility of the protease recognition site for the protease is impaired upon binding of the first (poly)peptide capable of binding the analyte and the analyte due to steric hindrance caused by the analyte to the protease. To achieve such an effect, it may be necessary to select the analyte such that it is bulky enough to impair accessibility of the protease recognition site. Accordingly, the analyte is preferably a (poly)peptide or protein or fragment thereof. “Adjacent to each other”, as used in this connection, means that the protease recognition site and the analyte binding site/first (poly)peptide capable of binding the analyte are directly linked to each other, with the protease recognition site being directly N-terminal or C-terminal of the analyte binding site/first (poly)peptide capable of binding the analyte, or linked by a short spacer comprising 1-20 amino acids, for example 1-5 amino acids, preferably 1-2 amino acids. The linkage occurs via covalent bonds, in particular peptides bonds, as the respective functional units (protease recognition site, analyte binding site and spacer) are all amino acid sequences.

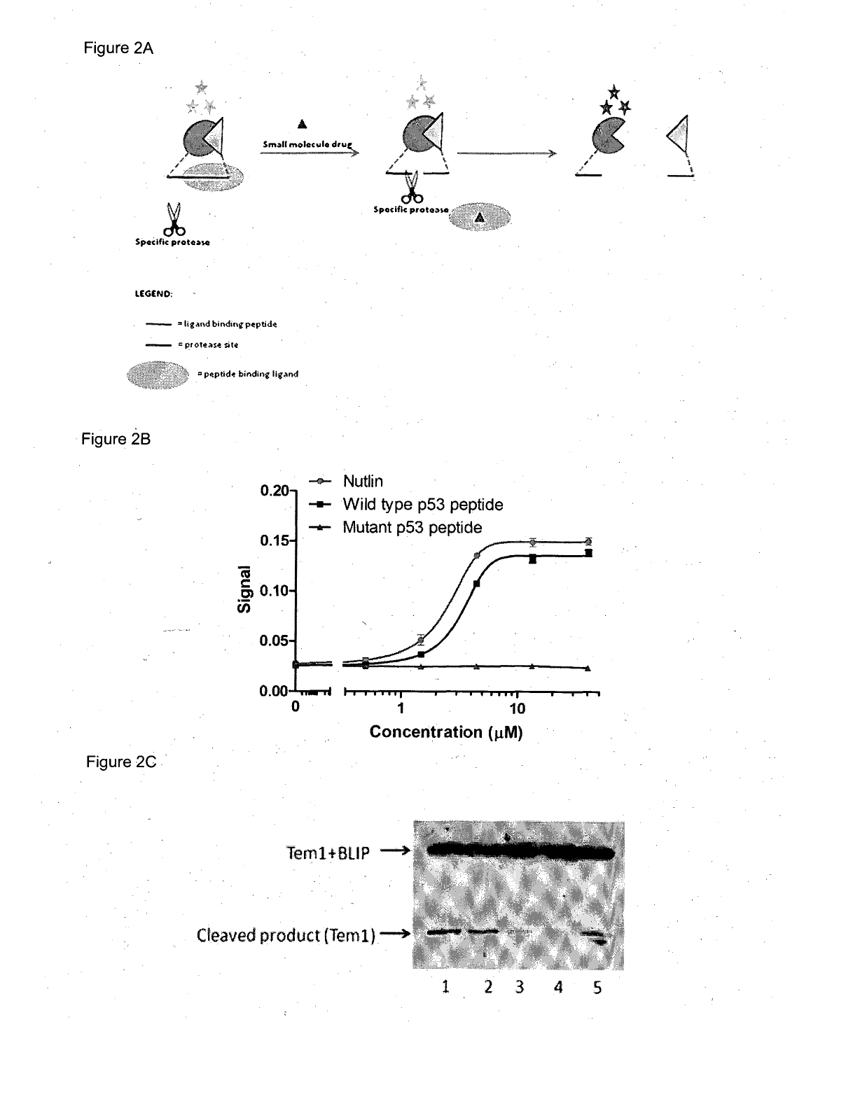

Since binding of the analyte to its cognate peptide will sterically hinder the protease from cleaving the protease recognition site, the signalling moiety will remain unaffected as long as the analyte is bound to the peptide biosensor. If the signalling moiety consists, for example of a fluorophore and a quencher, the fluorescence emission intensity of the fluorophore will be quenched in the protease responsive fusion protein/peptide by the quencher due to intramolecular quenching activity; however when the protease cleaves the protease responsive fusion protein/peptide at the protease recognition site, the fluorophore will be separated from the quencher and, as it is no longer quenched, the fluorescence emission intensity will increase. This may occur upon removal of the analyte, thereby restoring the protease access to the protease recognition site. This may allow screening for compounds that interfere with the analyte binding, as release of the analyte from the peptide biosensor in response to the analyte binding a candidate compound will restore the protease access to the protease recognition site and thus lead to signal generation, while in case the candidate inhibitor compound does not bind the analyte access to the protease recognition site will remain blocked for the protease. This detection/screening concept will be explained in more detail below and is schematically shown in FIGS. 2, 6A and 7A.

In some embodiments of the invention, in particular in embodiments where the protease recognition site and the analyte binding site/first (poly)peptide capable of binding the analyte are not arranged such that binding of the analyte to the analyte binding site reduces or prevents binding of the protease to the protease recognition site, the protease has a low affinity to the protease recognition site, for example by using an attenuated protease recognition site, or is prevented from binding the recognition site in the bulk solution by a competitive inhibitor, such that only in case the protease responsive fusion protein and the protease are both bound to the analyte in proximity to each other is the protease able to cleave the protease recognition site and release the moiety that can produce a detectable signal. “Attenuated” or “sub-optimal”, as used interchangeably in this context, means that the recognition site has been modified such that binding of the protease and cleavage is reduced compared to the non-attenuated form of the site. This may have the effect that the protease may only efficiently bind to and cleave the protease recognition site if the local concentration of both reaction partners, i.e. peptide biosensor and protease is sufficiently high. An increase of local concentration may for example be achieved by sequestering both the peptide biosensor and the protease to the same site such that they come in close proximity. Said sequestering may be done by providing a scaffold that provides binding sites for the peptide biosensor and the protease. The scaffold may be the analyte and the protease may, in such embodiments, be modified with an analyte binding molecule, such as a second (poly)peptide capable of binding the analyte. This analyte binding molecule/second (poly)peptide may be defined as the analyte binding site/first (poly)peptide above.

The proximity of the protease and the protease responsive peptide biosensor/fusion protein provides for enhanced cleavage at the protease recognition site leading to noticeable signal detection. It is understood that for recruiting the peptide biosensor and the protease to the analyte, the binding sites of the analyte binding site, e.g. the first (poly)peptide, and the analyte binding molecule, e.g. the second (poly)peptide, need to be different and allow simultaneous binding to the same analyte molecule. Alternatively, the analyte binding site, e.g. the first (poly)peptide, and the analyte binding molecule, e.g. the second (poly)peptide, may be the same, in which case the analyte needs to be multivalent, i.e. at least bivalent, for the respective binding partner. In these embodiments, the target binding sites on the analyte need to be sufficiently close to each other to allow bringing the peptide biosensor and the protease in close proximity to facilitate cleavage, i.e. so that the protease can noticeably recognize and cleave the protease recognition site at an accelerated rate, but sufficiently spaced apart to avoid impairment of the accessibility of the respective binding sites, i.e. still allow simultaneous binding of both.

In such embodiments, the local concentration of protease and protease recognition site is increased with the object of increasing the signal generated by the cleaved moiety capable of generating a detectable signal in the presence of the analyte.

In various embodiments, the peptide biosensor comprises or consists of the amino acid sequence set forth in any one of SEQ ID Nos. 6 and 18-21.

### Detection Reagents

To allow analyte detection and screening for compounds that interfere with the analyte binding to the peptide biosensor, the present invention also provides for detection reagents for the detection of an analyte. The detection of the analyte may be qualitative or quantitative, i.e. may in certain embodiments also include determining the amount of analyte. Analyte detection is preferable performed on a sample. The sample type is however not particularly limited, but preferably the sample is a biological sample, such as a cellular or body fluid sample.

The detection reagent includes the peptide biosensor as described above and a protease capable of binding to and cleaving the protease recognition site comprised in the peptide biosensor.

The protease may be selected from the proteases that have already been listed in connection with the description of the peptide and the protease recognition side above.

In various embodiments, the detection reagent comprises a peptide biosensor as described above, wherein the protease recognition site and the analyte binding site/first (poly)peptide capable of binding the analyte are not arranged such that binding of the analyte to the analyte binding site reduces or prevents binding of the protease to the protease recognition site. In such embodiments, the protease capable of binding to and cleaving the protease recognition site is coupled to an analyte binding molecule and the analyte is selected such that it can simultaneously be bound by the analyte binding site (of the peptide biosensor) and the analyte binding molecule (coupled to the protease) and wherein the protease recognition site and the protease are selected such that cleavage of the protease recognition site by the protease is detectably increased if both, the peptide biosensor and the protease, are bound to the analyte. In such embodiments, an attenuated protease recognition site, as has been described above, may be used. This attenuated protease recognition site may further increase the distinction between states where biosensor and protease are bound to the analyte and states wherein at least one of the two is not bound to the analyte.

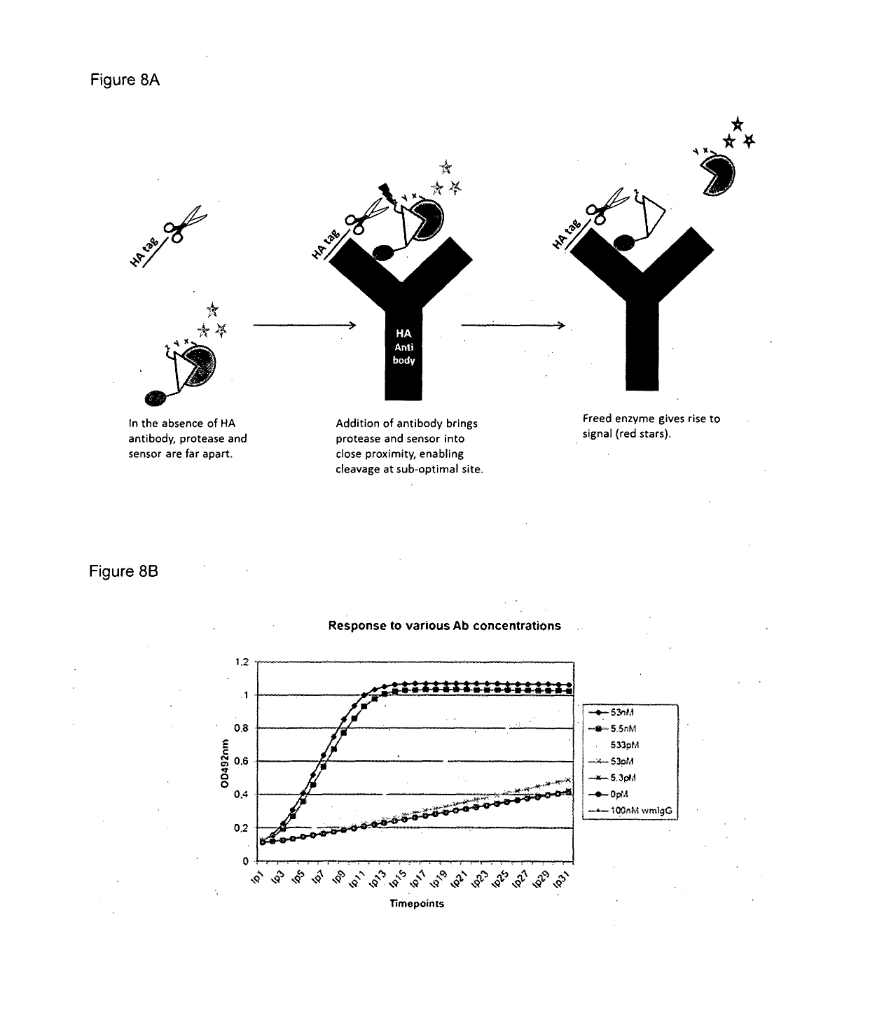

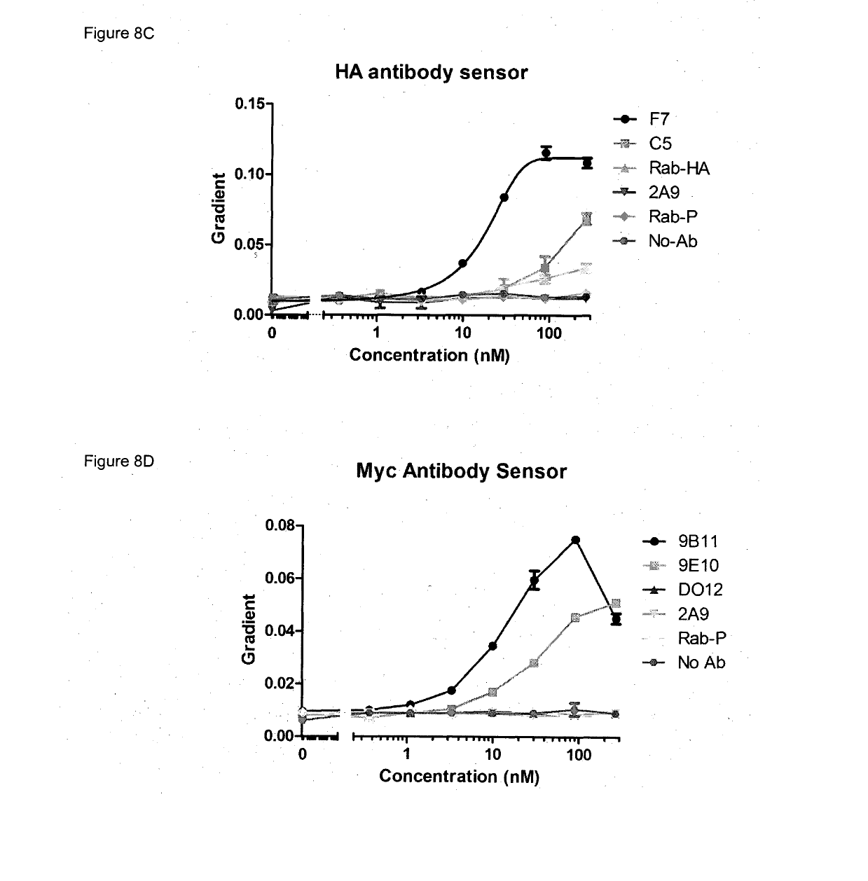

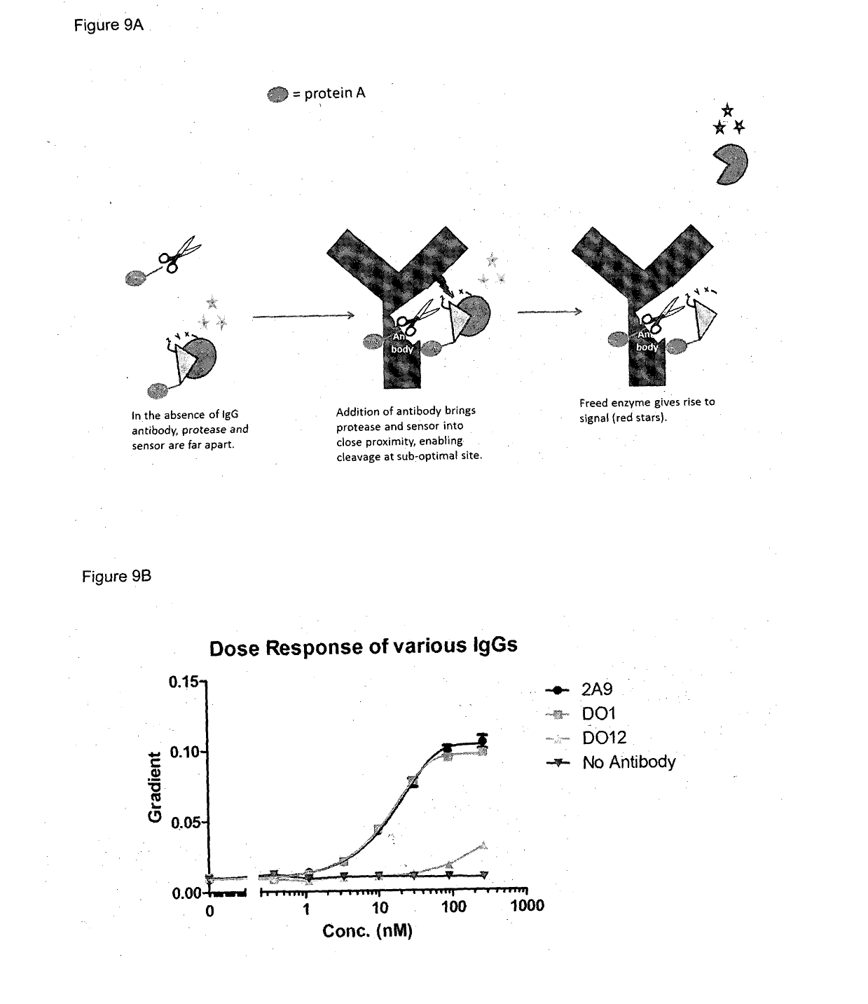

In such embodiments, the analyte binding molecule coupled to the protease may be a second (poly)peptide capable of binding to the analyte, preferably capable of specifically binding the analyte. Such embodiments are, for example, schematically depicted in FIGS. 8A and 9A.

As already described above, in certain embodiments the analyte binding site and the analyte binding molecule (e.g., the first and second (poly)peptide) may be the same, provided that the analyte is multivalent, i.e. a least bivalent, for said (poly)peptides capable of binding to the analyte. “Multivalent” or “bivalent”, as used herein, means that a given molecule or molecule complex has a least two binding sites for a given binding partner, so that two molecules of said given binding partner can bind to one given molecule or molecule complex. A typical example for such a bivalent analyte would be an antibody having two identical antigen-binding regions.

In other embodiments, the analyte binding site and the analyte binding molecule (e.g., the first and second (poly)peptide) are different and bind to different binding sites, wherein the binding sites for the analyte binding site and the analyte binding molecule are selected such that upon binding of the analyte binding site and the analyte binding molecule, the spatial proximity of peptide biosensor and protease is close enough to allow cleavage of the protease recognition site by the protease, while both are bound to the analyte, but far enough to avoid interference with the analyte binding of either.

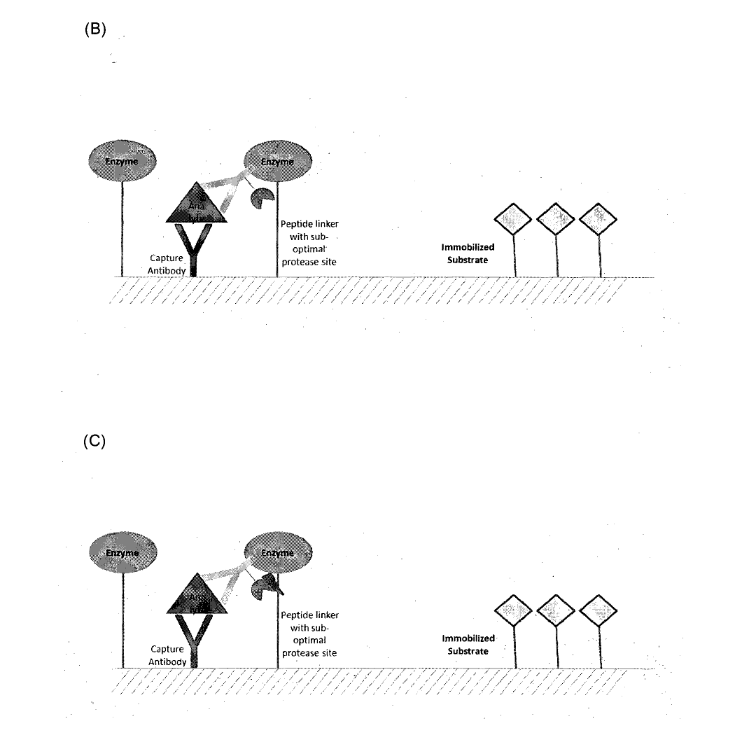

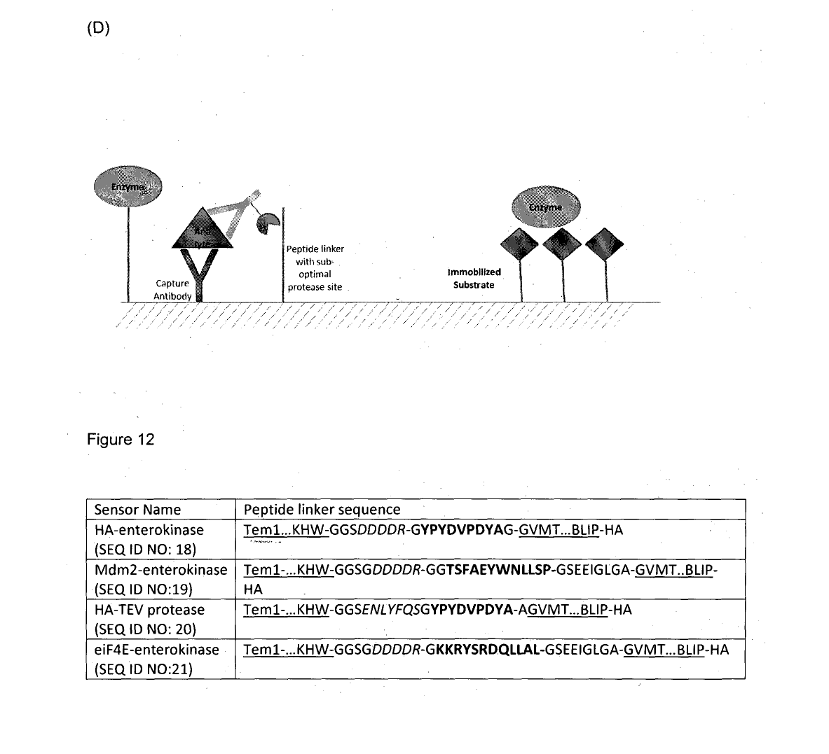

In certain embodiments of the invention, the detection reagent may also comprise a (solid) substrate onto which a peptide biosensor comprising a protease recognition site and a signaling moiety is immobilized such that upon cleavage of the protease recognition site by the protease, the signaling moiety gets released from the substrate. In such embodiments, the peptide biosensor does not comprise an analyte binding site, but a first (poly)peptide capable of binding the analyte is also immobilized on the substrate in proximity of the peptide biosensor. The detection reagent further comprises a protease coupled to a second (poly)peptide capable of binding the analyte, wherein both the first (poly)peptide immobilized on the substrate and the second (poly)peptide coupled to the protease can simultaneously bind to the analyte. The protease is not immobilized on the substrate but free in solution, and is recruited onto the substrate upon analyte binding by both the first (poly)peptide and the second (poly)peptide. With respect to the analyte binding, the assay thus resembles a sandwich (immuno)assay, in particular if both the first and second (poly)peptide are antibodies, with the first (poly)peptide being the capture antibody and the second (poly)peptide being the detection antibody. Recruiting the protease onto the substrate upon analyte binding, brings it in close proximity to the immobilized peptide biosensor so that the protease can bind to and cleave the protease recognition site, thus releasing the signaling moiety from the substrate surface. To allow cleavage of the protease recognition site by the protease, the peptide biosensor and the analyte capture reagent, i.e. the first (poly)peptide have to be immobilized on the substrate in close enough proximity. Releasing the signaling moiety leads to a detectable change in the generated signal which can be measured and used as an indicator for analyte binding. In this embodiment, the protease cleavage site, the signaling moiety, the first and second (poly)peptides and the protease may be defined as described above in relation to the other embodiments, with the main difference lying in that the peptide biosensor does not include the analyte binding site/first (poly)peptide but this is together with the peptide biosensor, but as a separate molecule, immobilized on a substrate. The substrate is preferably a solid substrate and may be any substrate known in the art and suitable for this purpose, such as for example the substrates used for immunoassays, such as ELISA, which typically are microtiter plates. A schematic depiction is shown in FIG. 11.

In these embodiments, the protease preferably has a low affinity to the protease recognition site, for example by using an attenuated protease recognition site, or is prevented from binding the recognition site in the bulk solution by a competitive inhibitor. The signaling moiety is preferably an enzyme and no enzyme modulator or inhibitor is used, but rather the substrate of the enzyme is immobilized on the same substrate or a different substrate such that it can only be converted by the enzyme once it is released from the substrate. This may for example be achieved by spacing the substrate far enough away from the enzyme such that conversion cannot occur as long as the enzyme remains immobilized. The set up of an assay using such detection reagent is schematically shown in FIG. 11.

In addition to the biosensor and the protease, the detection reagent may include any of numerous carriers and/or auxiliaries known in the art, including buffers, solvents, stabilizers, enzyme substrates (in case the signaling moiety comprises an enzyme) and the like. It is understood that such additional components may be selected based on the actual application and signal detection method and their selection is well within the routine capabilities of the skilled artisan in the field.

### Methods

In further aspects, the invention relates to methods of detecting the presence and/or amount of an analyte, preferably in a sample, said methods comprising: (i) contacting the detection reagent as described above with the analyte or a sample suspected of containing the analyte under conditions that allow binding of the analyte by the detection reagent; and (ii) detecting the presence and/or amount of the analyte in said sample by measuring the signal of the signaling moiety.

Further methods of the invention are directed to the screening for compounds that modulate the binding of an analyte to a binding partner of the analyte, said methods comprising: (i) contacting the detection reagent as described above with the analyte and a candidate compound under conditions that allow binding of the analyte by the detection reagent, wherein the analyte binding partner is the analyte binding site or the analyte binding molecule, optionally the first (poly)peptide capable of binding the analyte; and (ii) measuring the signal of the signaling moiety, wherein a decrease or increase in the signal compared to a reference not containing the candidate compound indicates modulation of the binding of the analyte and the analyte binding site or analyte binding molecule by the candidate compound.

The above methods may also comprise the step of providing the detection reagent prior to step (i).

As used herein, the term “contacting” may include to reacting or binding. Contacting may be bringing a compound and a target together such that the compound can affect the activity of the target. Contacting may be but is not limited to being performed in a test tube or a petri-dish. Contacting may involve incubation.

The term “detecting” refers to monitoring, determining, or sensing. The term “presence” may refer to the existence or a measureable level of the respective agent that is detected.

The term “under conditions” refers to being subject to a certain set of requirements or parametric control to achieve binding of the analyte molecule. For example, the conditions may be but is not limited to temperature and/or length of time.

As used herein, an “amount” may represent a measurable level.

The step of measuring the signal may include comparing the signal with a reference, such as a control measurement. The “control” measurement may be a positive or negative control measurement. The control measurement serves as a reference or basis against which comparison may be made to the detected measurement.

In various embodiments, the signal produced may be determined by fluorescence, absorbance, luminescence, enzymatic activity and the like.

In various embodiments, the method may be performed in a living cell in vivo or in vitro (ex vivo).

In the methods for screening for compounds, the detection reagents used preferably comprise the peptide biosensor as described above and comprising the protease recognition site, the analyte binding site and the signaling moiety and the protease that can cleave the protease recognition site. The peptide biosensor may be the one wherein the protease recognition site and the analyte binding site are positioned relative to each other such that analyte binding impairs protease-mediated cleavage of the peptide biosensor or, alternatively, the protease may be coupled to an analyte binding molecule, as described above.

The screening methods are particularly useful for identifying compounds, such as small molecule compounds, that interfere with the binding of the analyte to the analyte binding site/the first (poly)peptide capable of binding the analyte. Typically they compete with the binding of either of the two to the respective other partner and thus inhibit binding. Also possible if of course that they act as allosteric inhibitors and prevent analyte binding by inducing structural changes the impair the interaction between analyte and analyte binding site.

By binding to the analyte binding site or, preferably, the analyte and thus impairing or abrogating the binding of analyte binding site and analyte they can prevent binding or induce release of the analyte. In any case, the prevention of analyte binding or release of the analyte can the protease recognition site accessible for the protease and thus lead to signal generation (turn-on signal).

However, in case the other setup of the peptide biosensor is used where the detection principle relies on bringing the protease and the protease recognition site in close proximity by simultaneous binding of the protease and the biosensor to the analyte, the inhibitor would prevent this recruiting and no signal will be generated (turn-off signal).

Accordingly, for screening purposes the first approach relying on a turn-on signal is preferred.

In contrast, in methods where analyte detection is desired, the second approach is preferred, as the signal is turned on in presence of the analyte (since the analyte brings protease and peptide biosensor in close proximity).

Analyte detection may be performed on a sample, said sample being suspected of containing the analyte. The sample may be any sample type but is preferably a biological sample. Such a biological sample may be a cellular sample, with the cells being lysed or intact, or a biological fluid sample that may optionally contain intact cells.

### Kits

In a final aspect, the invention also relates to a kit for detecting the presence and/or amount of any analyte in a sample. Said kit comprises at least any one or more of the above described detection reagents. Additionally, the kit may contain instructions for use and/or the typical auxiliaries, such as buffers, detection reagents and the like. Accordingly, in various embodiments, the kits also comprise one or more substances that allow detecting and measuring the signal produced by the signaling moiety. In case the signaling moiety comprises an enzyme, these substances may include enzyme substrates.

In order that the invention may be readily understood and put into practical effect, particular embodiments will now be described by way of examples and not limitations, and, with reference to the figures.

### EXAMPLES

### Materials and Methods

Chemicals and reagents were purchased from Sigma Aldrich, unless indicated otherwise. The Mdm2 protein used here is derived from N-terminal domain (amino acids 18-125) of wild type Mdm2 protein and engineered with 10× His tag, The protein was expressed in E. coli and purified by immobilized affinity chromatography as described earlier (Brown et al.,2011, PloS One 6.8). The eukaryotic translation initiation factor 4E (eIF4E) was produced as previously reported (Brown et al.,2007, J. Mol. Biol. 372(1): 7-15). All synthetic peptides used were obtained from Bio-Synthesis, Inc (USA).

### Experimental Verification Using Fluorescence Labeled Peptide

The protease exclusion concept was first validated by using a peptide (P1) labeled with a fluorophore and quencher pair. The peptide sequence is as follows: E(EDANS)-SG DDDDR-GK (Dabcyl)-TSFAEYWNLLSP-GS (SEQ ID NO:6). In this peptide, the fluorophore 5-((2-Aminoethyl)amino)naphthalene-1-sulfonic acid (EDANS) is conjugated as a side chain to glutamic acid followed by amino acids SG as a linker. DDDDR (SEQ ID NO:1) is an enhanced enterokinase cleavage recognition site (Boulware and Daugherty, 2006, PNAS USA 103 (20): 7583-7588). The EDANS quencher 4-(dimethylaminoazo)benzene-4-carboxylic acid (Dabcyl) is conjugated to the side chain of lysine followed by a high affinity peptide sequence (TSFAEYWNLLSP; SEQ ID NO:3) derived by phage display which binds the p53 binding pocket in the Mdm2 N-terminal domain. In a final volume of 25 ml with 4% DMSO buffered by phosphate buffered saline (PBS) at pH. 7.3, 1.33 mM fluorescent labeled peptide (E(EDANS)-SG DDDDRGK (Dabcyl) TSFAEYWNLLSP GS), 152 pM enterokinase and 3.2 mM Mdm2 are present. Apart from these constant reagents, triplicates of varying concentrations of Nutlin, wild type p53 peptide (ETFSDLWKLLS; SEQ ID NO:7) and a mutant p53 peptide with critical residues mutated to alanine (ETASDLAKLAP; SEQ ID NO:8) were added to the reaction. Enterokinase was added last, after a 5 min delay to ensure that no premature cleavage of peptide occurs. The resulting reaction was read on a Perkin Elmer plate reader in a 384 well black bottom plate (Greiner) with excitation at 335 nm and emission at 490 nm. Readings were taken every 5 min. The signal was calculated as the difference between emission intensity at reading 3 and 5. Every experiment was repeated three times.

### Plasmid Construction and Oligonucleotides

Four plasmids were used in this invention. HA-enterokinase plasmid was constructed by inverse PCR (Nirantar et. al., 2013, Biosens. Bioelectron. 47(0): 421-428) of a codon optimized Tem1-BLIP (D49A) cassette (Genscript) placed in the NdeI XhoI sites of pET28a using the oligonucleotides 1 and 2 as described in Table 1. Oligo 1 is a Tem1 reverse oligo whose 5′ partially codes for the intended linker sequence. Oligo 2 is a BLIP forward oligo whose 5′ has 15 bases complementary to oligo 1 for infusion cloning purposes, and codes for the rest of the peptide linker sequence. After the inverse PCR, the PCR product was treated with Dpnl, purified and treated using the infusion cloning enzyme (Clontech) to enable intra-molecular infusion, followed by transformation in JM109HIT competent cells (RBS Biosciences).The HA-TEV plasmid was made as above, using oligonucleotides 3 and 4 (Table 1), using the HA-enterokinase plasmid as a template. The Mdm2-enterokinase sensor was constructed in the same way, except that the Tem1-BLIP (D49A) template used has a linker present between Tem 1 and BLIP. Oligonucleotide 5 (Table 1) is a reverse oligo, while tandem oligonucleotides 6 and 7 are forward oligos complementary to the linker sequence. Tandem oligonucleotides were used due to the length of the peptide linker to be inserted. The eiF4E-enterokinase sensor was made as the Mdm2-enterokinase sensor, using oligonucleotide 5 as a reverse oligo and oligonucleotides 8 and 9 as tandem forward oligos (Table 1).

The HA-Thrombin plasmid was constructed by doing inverse PCR with a Tem1-BLIP template using oligos 10 and 11, followed by Dpnl treatment, PCR purification and linkage of the ends of the PCR product by infusion cloning followed by transformation in competent cells.

TABLE 1

| Oligo | Sequence |
|---|---|
| 1) | AACATCGTACGGATAGCCACGGTCAT |
| | CGTCATCGCTACCGCCCCA |
| | (SEQ ID NO: 9) |
| 2) | TATCCGTACGATGTTCCGGACTACGC |
| | CGGAGGTGTT |
| | (SEQ ID NO: 10) |
| 3) | CTGAAAATACAGGTTTTCGCTACCGC |
| | CCCAATGTTT |
| | (SEQ ID NO: 11) |
| 4) | AACCTGTATTTTCAGTCTGGCTATCC |
| | GTACGAT |
| | (SEQ ID NO: 12) |
| 5) | ACGATCGTCATCGTCACCACTACCGC |
| | CCCAATG |
| | (SEQ ID NO: 13) |
| 6) | GACGATGACGATCGTGGTGGTACTAG |
| | CTTTGCAGAATATTGG |
| | (SEQ ID NO: 14) |
| 7) | GCAGAATATTGGAACCTGTTGTCTCC |
| | GGGATCCGAAGAGATT |
| | (SEQ ID NO: 15) |
| 8) | GACGATGACGATCGTGGTGGTAAAAA |
| | GCGTTATAGCCGTGAT |
| | (SEQ ID NO: 16) |
| 9) | TATAGCCGTGATCAACTGTTAGCGCT |
| | GGGATCCGAAGAGATT |
| | (SEQ ID NO: 17) |
| 10) | TATCCGTACGATGTTCCGGACTACGC |
| | CGGAGGTGTT |
| | (SEQ ID NO: 22) |
| 11) | AACATCGTACGGATAaccacgcggaa |
| | ccagaccgctACCGCCCCA |
| | (SEQ ID NO: 23) |

### Sensor Protein Production

The relevant plasmid was transformed into SHuffle T7 Express Competent Escherichia coli cell (New England Biolabs), the transformed cells were grown over night in LB medium containing 50 μg/ml kanamycin sulfate at 30° C. under shaking conditions. 1% (v/v) of the over night culture was inoculated into 250 ml LB medium at 30° C., and protein expression of the fusion proteins was induced with IPTG at OD600=0.7-0.8. After 4 h post-induction at room temperature, the cells were harvested by centrifugation. The washed cell pellets were resuspended in 20 ml 50 mM phosphate buffer (pH 7.4) and disrupted by sonication using a digital sonifier (LifeTechnologies, Novex). Sonication was performed for 15 cycles at 5 s/cycle, followed by 10 s cooling after each sonication cycle. The lysed cells were centrifuged at 10,000 g for 30 min, and the suspension was then collected. The filtered supernatant of cell lysate containing sensor protein with a 6× His tag was loaded onto, a 1 ml Ni Sepharose His Trap column (GE Healthcare). The column was pre-equilibrated with Buffer A (50 mM phosphate buffer, 300 mM NaCl, 30 mM imidazole, pH 7.4). After washing with 10 column volume (CV) of Buffer A, the target sensor protein was eluted at 80% Buffer B (phosphate buffer, 300 mM NaCl, 500 mM imidazole, pH7.4) over 15 CVs. The recovered sensor protein was stored in −80° C. for further usage.

### HA-Enterokinase Sensor Assay

All reactions were performed in 25 μl PBS with 250 mM substrate (Nitrocefin, Merck) and Greiner Bio 384 well transparent bottom plates were used to hold the reactions. The reaction was monitored using absorbance measurements at OD492 on a Perkin Elmer plate reader every 2 min. The sensor response to enterokinase was first investigated by mixing 5 nM relevant sensors (diluted from 1.25 mM stock) with various amount of enterokinase (0.3 nM-1.2 nM) at room temperature; TEV (1.2 nM) was used as the negative control. Subsequently, the sensor response to anti-HA antibody F-7 (Santa Cruz Biotech) was investigated by adding various amount of HA antibody (3 nM to 10 nM) in the presence of 1.2 nM enterokinase; 10 nM whole mouse IgG was used as the control. Sensor response to various concentrations of free HA peptide (0.8 nM to 8 mM) was carried out in the presence of 1.2 nM enterokinase and 10 nM anti-HA antibody, 8 mM p53 peptide was used as the control. The rate of substrate turnover, typically the OD492 value of read number 3 subtracted from that of read number 5 (OD492 readings were taken every 2 min) was denoted as the signal. Every experiment was repeated three times and the average with standard error was reported.

### Mdm2-Enterokinase Sensor Assay

The reaction was carried out in 25 μl PBS with 5 nM sensor, 0.15 nM enterokinase, 40 nM Mdm2 and 250 μM nitrocefin. Nutlin was used as the positive control and a mutant p53 peptide, which cannot bind to Mdm2 was used as the negative control. The reaction was monitored using absorbance measurements at OD492 on a Perkin Elmer plate reader every 2 min. The signal was calculated from reading 5 minus reading 3. This assay was repeated three times and the average gradient value with standard error was reported.

eiF4E-Enterokinase Sensor Assay:

The reaction was carried out in 25 μl PBS with 5 nM sensor, 0.15 nM enterokinase, 5 μM eiF4E protein and 250 μM nitrocefin. Various concentrations of free eiF4E peptide were added and the system was monitored using OD492 as above. The signal was calculated as the difference between reading 5 and reading 3. The assay was repeated in triplicate.

### Mdm2-Binding Fragment Discovery Using Mdm2-Enterokinase Sensor

The Zenobia fragment library containing 352 compounds (Zenobia Therapeutics, San Diego, USA; Fragment Library 1) was used to test the performance of p53-Mdm2 sensor in high throughput screening (Chessari and Woodhead, 2009, Drug Discovery Today 14(13-14):668-675). The screening was divided into two steps. In the first step, the screening was carried out at the fixed compound concentration of 800 mM. In the second step, a dose-response experiment of different hits found in the first step was conducted. The reaction was carried out in 25 ml PBS with 5 nM sensor, 0.15 nM enterokinase, 40 nM Mdm2, 3% DMSO and the compounds were varied from 800 mM to 9.8 mM. The OD492 value of read number 3 was subtracted from that of read number 5 (OD492 readings were taken every 2 min) which was designated as signal. These gradients were divided by the gradient of negative control to give the fold change. This data is represented as fold change (Y axis) vs. compound concentrations (X axis). Fold change=Gradient of Hits (Reading 5−Reading 3)/Gradient of control (Reading 5−Reading 3)

### Fluorescence Polarization

Fluorescence polarization measurements were performed essentially as described in previous study (Brown et al., 2013, ACS Chem. Biol. 8 (3): 506-512). Briefly, in a 100 ml reaction buffered by 0.1% PBS-Tween (pH 7.3), 50 nM of fluorescently labeled p53 derived 12-1 peptide (FAM-RFMDYWEGL-NH2) which can bind to Mdm2 and 200 nM Mdm2 were added, along with the indicated amounts of competing antagonists. Reactions were carried out in duplicate and the average with standard error was reported. The fluorescence polarization values were determined using a Perkin Elmer plate reader at the excitation/emission wavelengths of 485/535 nm.

Mdm2-p53 Immunoprecipitation and qPCR

The immunoprecipitation of p53 via Mdm2 was carried out essentially as described before (Wei et al., 2013, PloS One 8 (4), e62564). Briefly, full-length Mdm2 and p53 were synthesized separately in in vitro translation (IVT) mix, followed by capture of the Mdm2 on magnetic beads via an HA tag. Indicated amounts of the relevant drugs were then added for 1h followed by IVT synthesized p53 for an additional hour. The complex was washed 6 times followed by Western blotting to detect p53. Measurement of p53-Mdm2 interaction by qPCR was carried out as previously described (Wei et al., 2013, supra), with the exception that Mdm2 N-terminal domain (amino acids 1-125) was used as bait.

### Example 1

### HA-Protease Exclusion Sensor

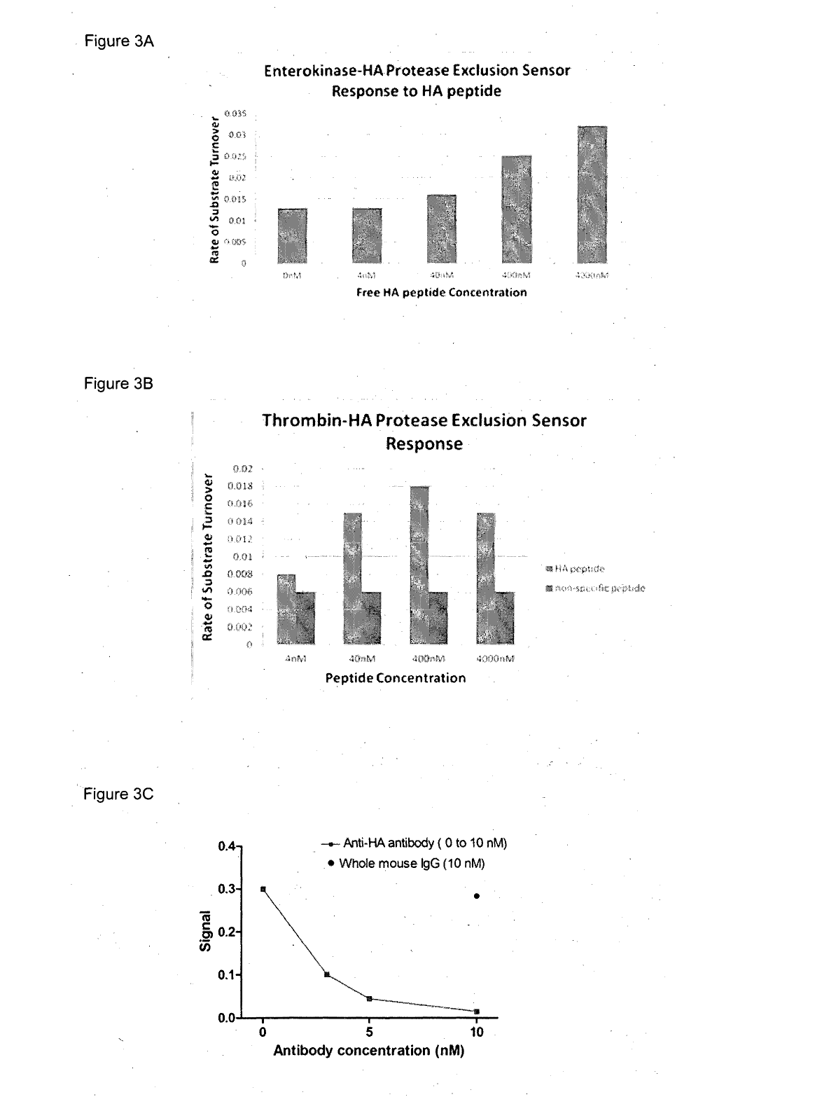

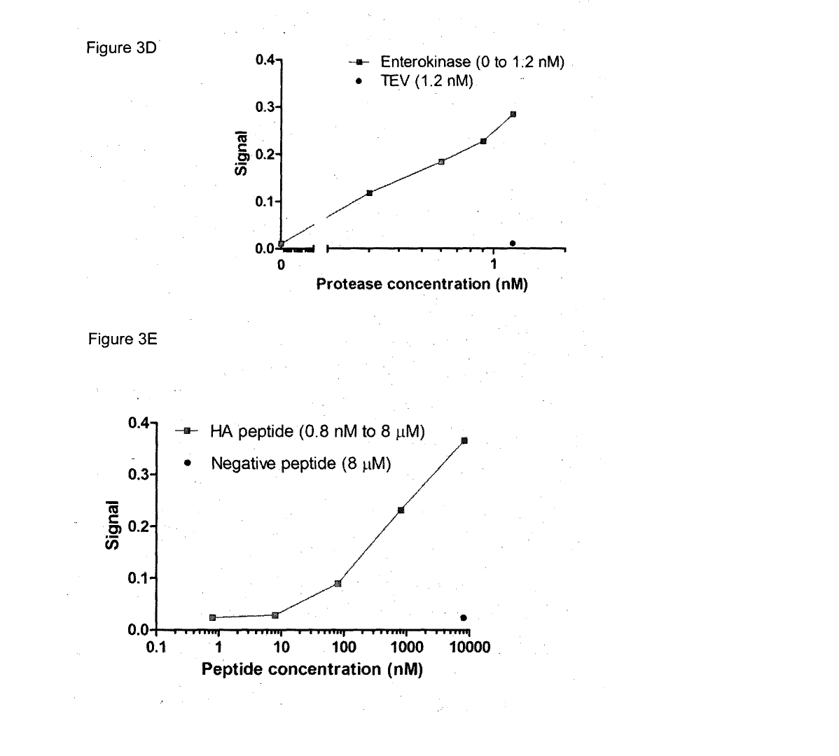

The sensor, underlying the general concept as shown in FIG. 2, involves the configuration Tem1-protease site-HA epitope-BLIP, while the spacing between protease and HA-epitope is kept short (one glycine residue). An HA-antibody binds to the HA epitope (YPYDVPDYA; SEQ ID NO:3). The Binding of an anti-HA antibody to its epitope would disallow protease, access to its recognition site due to steric obstruction by the higher affinity HA antibody. Addition of excess HA peptide should sequester the HA antibody, leading to renewed protease access to its recognition site, bringing about increased enzyme activity in response to the HA peptide. Testing of this sensor shows a concentration-dependent increase of substrate turnover after addition of HA-peptide of an enterokinase-HA protease exclusion sensor, as depicted in FIG. 3A. Starting from the left, the rate of substrate turnover is shown after 0 nM, 4 nM, 40 nM, 400 nM and 4000 nM addition of free HA peptide to the tested sensor.

FIG. 3B shows the highest substrate turnover after addition of 400 nM HA peptide for a thrombin-HA protease exclusion sensor. Substrate turnover after adding a non-specific substrate was constant, independent of the amount of peptide.

FIG. 3C shows the decrease of substrate turnover specifically upon increasing amounts of HA-antibody. The graph shows substrate turnover signals upon 0 nM to 10 nM anti-HA antibody (squares) and 10 nM whole mouse IgG as a control, indicated by the closed circle.

FIG. 3D shows that the signal of substrate turnover also increases with the amount of protease. In the graph, from left to right signals after 0 nM to 1.2 nM enterokinase (squares) are shown. An excess of 1.2 nM TEV protease was added as a control (closed circle).

### Example 2

### Enterokinase Mdm2 Protease Exclusion Sensor

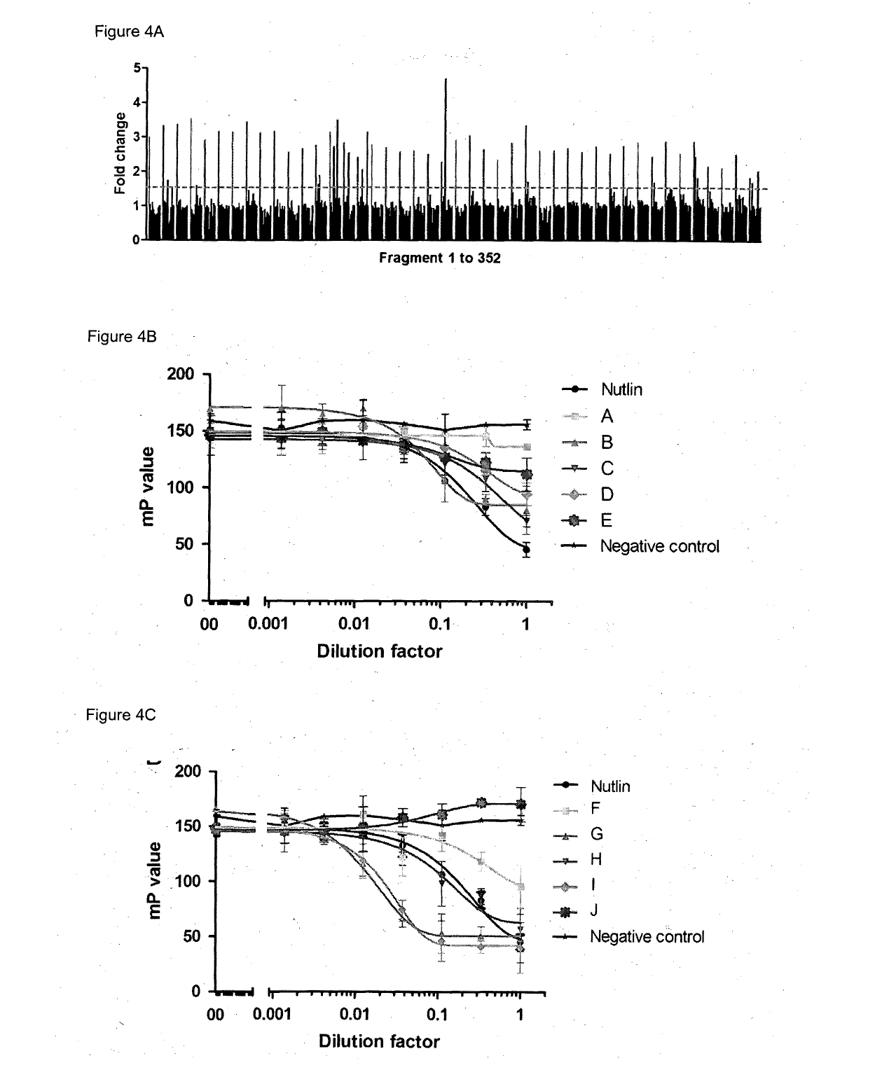

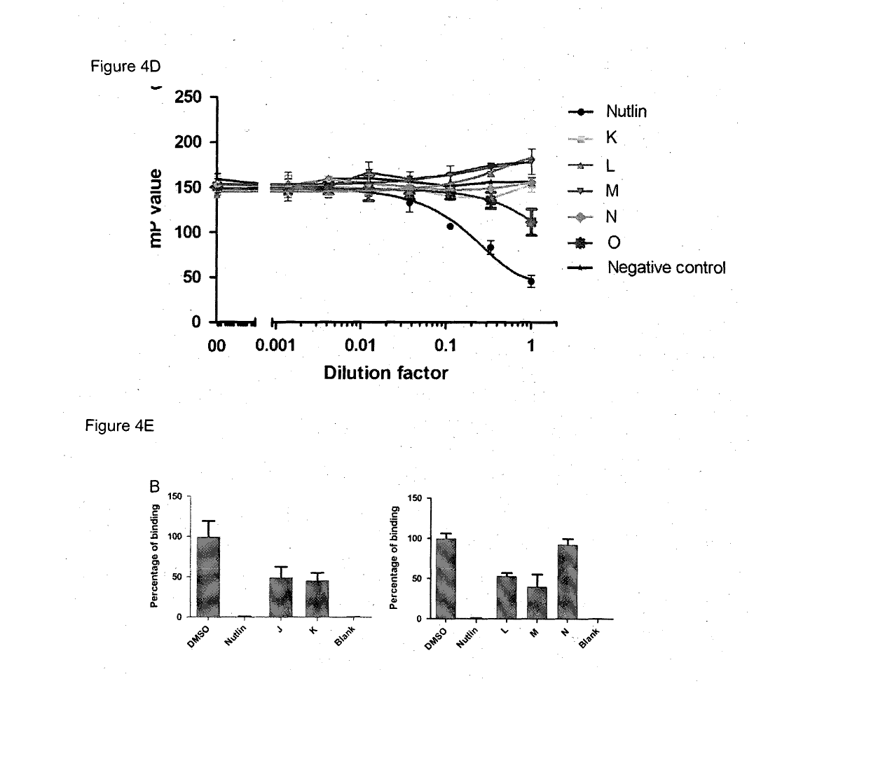

Testing of a sensor wherein a p53 peptide was placed adjacent to an enterokinase site in a peptide linker between Tem1 and BLIP are shown in FIGS. 4B-D. FIG. 4B shows a time course of a treatment assay of this sensor with enterokinase and mdm2 (which binds the p53 peptide placed next to the enterokinase site). The presence of solvent DMSO only leads to low substrate turnover (squares). However, when Nutlin (an antagonist of the mdm2-p53 peptide interaction as shown in FIG. 4A) or a free p53 peptide is added to this reaction, substrate turnover increases substantially (indicated by the blue signs). Treatment with non-specific moieties such as 5 FU (a small molecule) or a mutant p53 peptide (peptide C; does not bind mdm2 N terminus) does not lead to increased turnover, in accordance with the predictions of the protease sensor concept.

In FIGS. 4C and 4D the response upon using various competitors of Mdm2 are shown. Both graphs shows the rate of turnover of substrate in response to various amounts of free peptides. Responses to Nutlin (Nutlin 3a in FIG. 4D), wildtype (WT) p53 peptide are increased turnover signal (upper lines) in contrast to the mutant p53 peptide (triangles).

For testing further competitor compounds for Mdm2, a drug screening application by interrogating a small (n=352) library of low molecular weight compounds for Mdm2 antagonists were used (Fragment library 1 containing 352 compounds; commercially available from Zenobia Therapeutics, San Diego, USA;). In the primary screen, 15 hits were detected as shown in FIG. 4H that successfully inhibited Mdm5-p53 interaction.

FIGS. 4E to 4G show tested interaction between Fragments from the screen A to O and Mdm2 N-terminus in a competitive fluorescence polarization assay. Nutlin was used as a positive control (closed circles) and a non-reactive fragment (stars) as a negative control.

### Example 3

### Enterokinase eiF4E Protease Exclusion Sensor

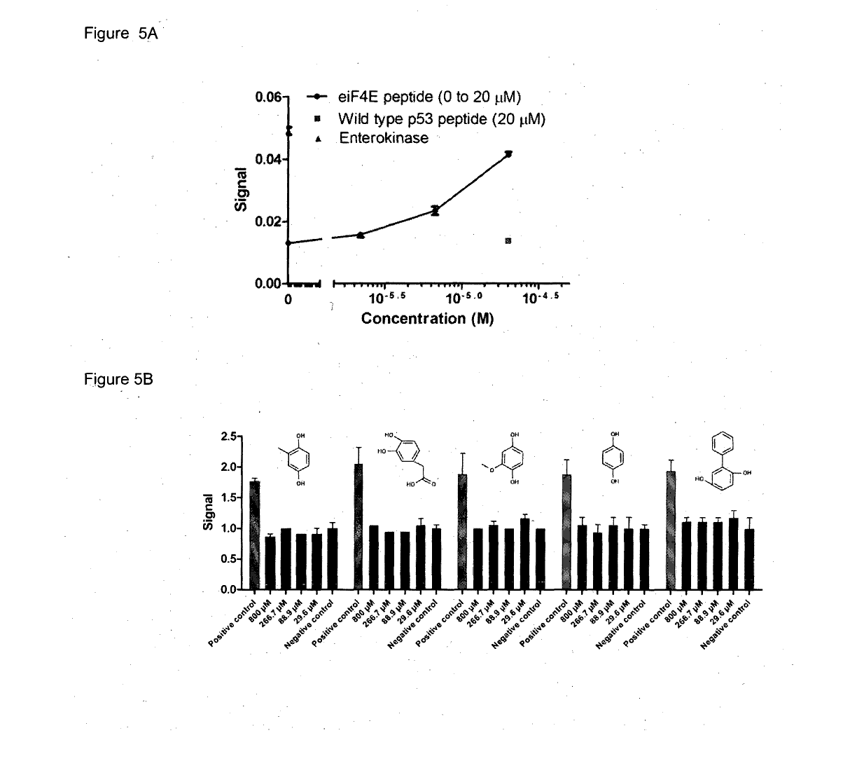

FIG. 5A shows the response of eiF4E-enterokinase sensor treated with eiF4E protein and enterokinase, along with various concentrations (0 nM to 20 nM) of free eiF4E peptide (full circles). The rate of substrate turnover is plotted on the Y axis. The rate of turnover seen in the absence of eiF4E protein is shown as the black data point n the Y axis. As control, a non-specific wild type p53 peptide (squares) at the highest concentration used for the eiF4E peptide was also assayed.

FIG. 5B shows the dose response of eiF4E-enterokinase sensor to the 5 best hits (from left to right shown fragments B, D, G, I and K) obtained from the Mdm2-enterokinase sensor primary screening as described in Example 2. Each Fragment was tested in concentration of 29.6 μM, 88.9 μM, 266.8 μM and 800 μM. The first bar of each set indicates a positive control using Nutlin, and the last bar shows a negative control.

### Example 4

### Protease Exclusion Sensor Using a Synthetic Internally Quenched Peptide

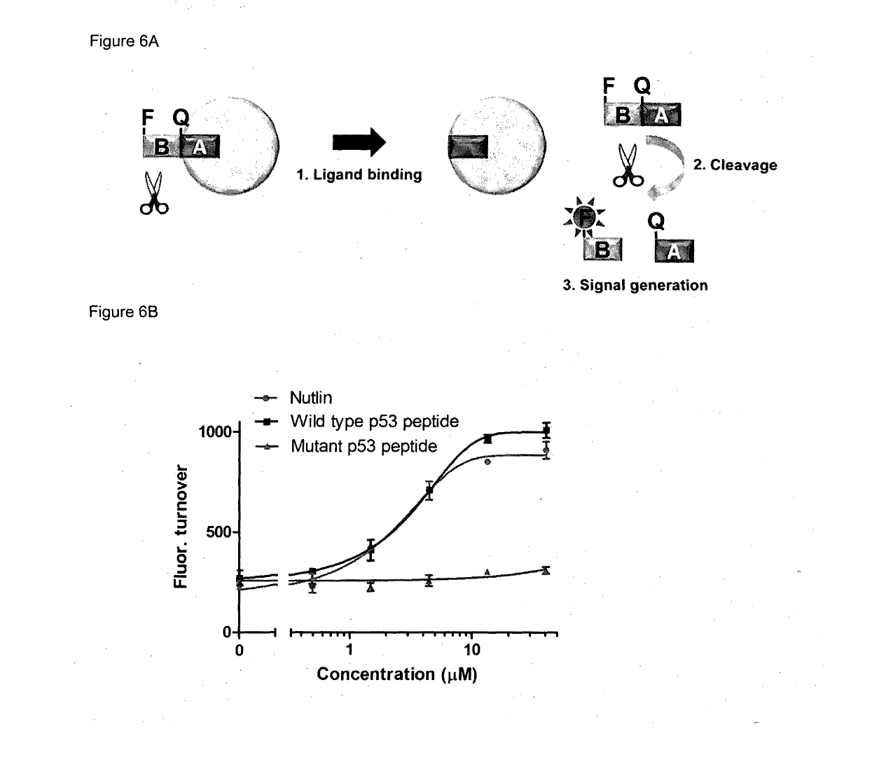

As shown in FIG. 6A, the peptide used herein has a fluorophore (EDANS, shown as a star) at the N terminus, followed by a protease site (DDDDR), a single amino acid glycine linker, a quencher (small circle) attached to a lysine side chain of the N-terminal side of a p53 based peptide sequence which binds the Mdm2 N-terminus. As the fluorophore s placed close to a quencher, the emission intensity will be low. Cleavage of the peptide by enterokinase (¾th circle) between the fluorophore and quencher will lead to a sharp increase in the fluorescence intensity. Upon incubation with purified ‘Mdm2 N-terminus domain (oval), it binds to the p53 derived peptide segment. This hinders enterokinase access to its protease site due to steric clashes with Mdm2 N-terminus, preventing an increased fluorescent signal. Addition of a small molecule drug (triangle) or a peptide which is a competitive inhibitor of the Mdm2-p53 peptide interaction sequesters the Mdm2 N-terminus, thereby restoring enterokinase access to its protease site, causing increased fluorescence (shown as increased size of the star).

FIG. 6B shows the testing of various concentrations of Nutlin (full circles), WT (squares) and mutant p53 peptide triangles). The peptide configuration shows a strong response to Nutlin and the WT peptide but not the non-binding mutant peptide.

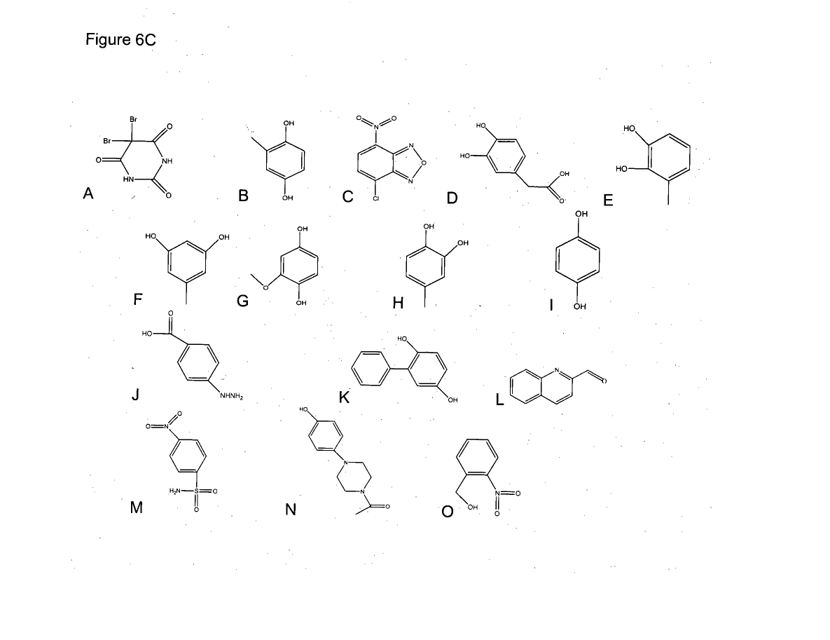

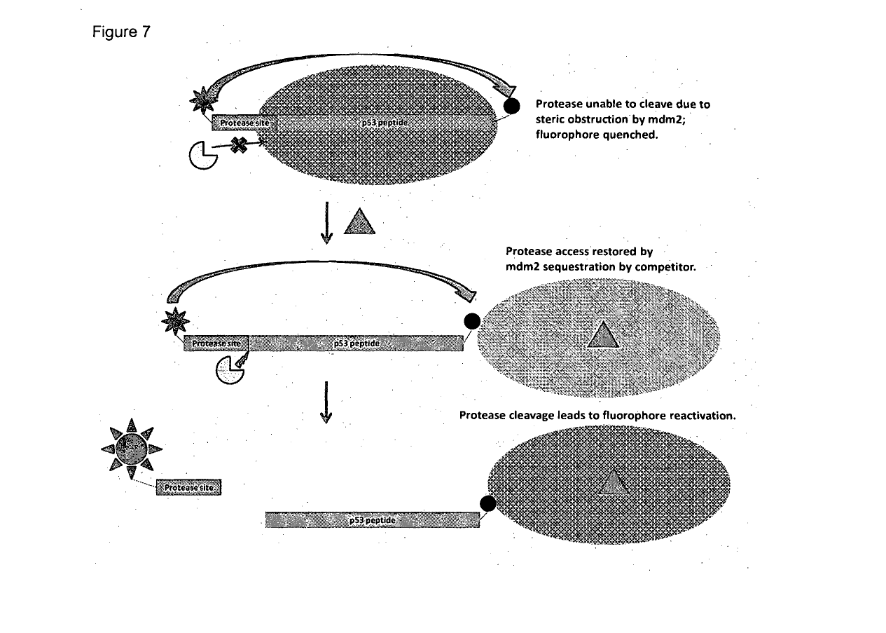

FIG. 7 shows a similar protease exclusion sensor wherein the quencher was moved to the C-terminus of the p53 based peptide. The increased distance between fluorophore and quencher in this peptide leads to less efficient quenching, but also allows more steric obstruction of the enterokinase site by Mdm2 N-terminus due to the closer spacing between the Mdm2 N-terminus and the protease site.

### Example 5

### Protease Exclusion Sensor Using Enhanced Protease Signaling Concept

The protease and its sensor are fused to moieties that recognize an analyte. When the analyte is introduced, these moieties bind, thus bringing the protease and its sensor into close proximity. This leads to a much greater effective concentration of both sensor and protease, enabling the protease to cleave the linker despite the suboptimality of the site, leading to signal generation. Thus the enhanced cleavage of the linker, facilitated by the analyte, leads to signal generation proportional to the amount of analyte present. As the schematic depiction in FIG. 8A shows that an HA-antibody was used as an analyte.

FIG. 8B shows a large increase in signal generation proportional to the HA-antibody (analyte) amount is seen. Here, protease and the sensor (as in FIG. 8A) fused to protein L which binds antibody light chains was expressed and the fluorescent signal was measured at OD492 nm. Antibody concentrations of 0 pM, 5.3 pM, 53 pM, 533 pM, 5.5 nM and 53 nM between time points 1 and 31 are depicted.

FIG. 8C shows the results of testing different HA-antibodies (F7, C5 and Rab-HA) as analyte in a sensor as described above. Testing the signal with unspecific antibodies 2A9 and Rap-P and testing without an antibody was used as controls. The F7-HA antibody showed the highest signal.

FIG. 8D shows a similar test for a myc antibody sensor, comparing 9B11, 9E10 and DO 12. Controls were used as described for FIG. 8C.

FIG. 9A is based on the same concept as depicted in FIG. 8A and described above, except that protease and the sensor are fused to protein A. This leads to a binding of the protease and the sensor to the heavy chain of the antibody.

FIG. 9B shows the dose response of various tested IgGs, indicating that 2A9 and DO1 showed an increase of the signal upon elevating antibody concentration. This was not the case for the tested DO12 IgG. The gradient was also measured without using an IgG as a negative control.

The listing or discussion of a previously published document in this specification should not necessarily be taken as an acknowledgement that the document is part of the state of the art or is common general knowledge. All documents listed are hereby incorporated herein by reference in their entirety.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1-27. (canceled)

28. Peptide biosensor for the detection of an analyte, wherein the peptide biosensor comprises
(a) a protease recognition site;
(b) an analyte binding site; and
(c) a signaling moiety that can produce a detectable signal upon cleavage of the protease recognition site by a protease,
wherein the protease recognition site and the analyte binding site are positioned relative to each other such that binding of the analyte to the analyte binding site reduces or prevents binding of the protease to the protease recognition site, or wherein the protease recognition site is an attenuated protease recognition site.

29. The peptide biosensor of claim 28, wherein the peptide biosensor is a fusion protein.

30. The peptide biosensor of claim 28, wherein the analyte binding site is a first (poly)peptide capable of binding the analyte, preferably capable of specifically binding the analyte.

31. The peptide biosensor of claim 28, wherein the signaling moiety comprises or consists of one or more (poly)peptide(s).

32. The peptide biosensor of claim 28, wherein the signaling moiety comprises or consists of a signal generating moiety and a modulator, wherein the modulator when bound to the signal generating moiety modulates the signal generation by the signal generating moiety and wherein cleavage of the protease recognition site by a protease interferes with the binding of the modulator to the signal generating moiety.

33. The peptide biosensor of claim 32, wherein the signal generating moiety and the modulator each independently comprises or consists of a (poly)peptide.

34. The peptide biosensor of claim 32, wherein the signal generating moiety is a (poly)peptide coupled to a substance that can produce the detectable signal, with said substance optionally being a fluorophore or chromophore, or wherein the signal generating moiety is an enzyme or has enzymatic activity, with the modulator preferably being an activator or inhibitor of said enzyme or said enzymatic activity.

35. The peptide biosensor of claim 32, wherein the modulator is a (poly)peptide coupled to a substance that can modulate the detectable signal produced by the signal generating moiety, with said modulator substance optionally being a quencher for a detectable signal producing substance that is fluorophore or chromophore.

36. The peptide biosensor of claim 32, wherein
(a) the signal generating moiety comprises or consists of an enzyme and the modulator comprises or consists of an inhibitor of said enzyme; or
(b) the signal generating moiety comprises or consists of a fluorophore or chromophore and the modulator comprises or consists of a quencher of said fluorophore or chromophore.

37. The peptide biosensor of claim 28, wherein the peptide biosensor is a fusion protein comprising in N- to C-terminal orientation a structure selected from the group consisting of structures (I)-(VIII):
B-P-C-A (I);
C-P-B-A (II);
C-A-P-B (III);
C-P-A-B (IV);
B-A-P-C (V);
B-P-A-C (VI);
A-B-P-C (VII); or
A-C-P-B (VIII)
wherein
A represents a (poly)peptide capable of binding the analyte, preferably capable of
specifically binding the analyte;
B represents a signal generating moiety that can generate a detectable signal;
C represents a modulator that is capable of binding to and modulating the signal generation by B;
P represents the protease recognition site; and
“-” represents a covalent bond or a peptide linker comprising or consisting of one or more amino acids.

38. The peptide biosensor of claim 37, wherein the extent of the modulation of the signal generation by B by the modulator C detectably varies between the cleaved and non-cleaved state of the peptide biosensor.

39. Detection reagent for the detection of an analyte, wherein the detection reagent comprises
(1):
(a) a peptide biosensor comprising
a protease recognition site;
an analyte binding site; and
a signaling moiety that can produce a detectable signal upon cleavage of the protease recognition site by a protease,
wherein the protease recognition site and the analyte binding site are positioned relative to each other such that binding of the analyte to the analyte binding site reduces or prevents binding of the protease to the protease recognition site, or wherein the protease recognition site is an attenuated protease recognition site; and
(b) a protease capable of binding to and cleaving the protease recognition site; or
(2):
(A) a solid substrate comprising immobilized thereon:
(a) a peptide biosensor comprising
(i) a protease recognition site; and
(ii) a signaling moiety,
wherein the peptide biosensor is immobilized such that upon cleavage of the protease recognition site by a protease, the signaling moiety gets released from the substrate.
(b) a first (poly)peptide capable of binding the analyte, wherein said first (poly)peptide is immobilized on the substrate in proximity of the peptide biosensor; and
(B) a protease coupled to a second (poly)peptide capable of binding the analyte, wherein both the first (poly)peptide immobilized on the substrate and the second (poly)peptide coupled to the protease can simultaneously bind to the analyte.

40. The detection reagent of claim 39, wherein the detection reagent comprises (a) the peptide biosensor and (b) the protease capable of binding to and cleaving the protease recognition site, wherein said protease is coupled to an analyte binding molecule and wherein the analyte can simultaneously be bound by the analyte binding site and the analyte binding molecule and wherein the protease recognition site and the protease are selected such that cleavage of the protease recognition site by the protease is detectably increased if both, the peptide biosensor and the protease, are bound to the analyte.

41. The detection reagent of claim 40, wherein the analyte binding molecule coupled to the protease is a second (poly)peptide capable of binding to the analyte, preferably capable of specifically binding the analyte.

42. The detection reagent of claim 40, wherein the first and second (poly)peptide are the same and the analyte is at least bivalent for said (poly)peptides capable of binding to the analyte, or wherein the first and second (poly)peptide are different and bind to different binding sites, said binding sites for the first and second (poly)peptide being selected such that upon binding of the first and second (poly)peptide the proximity is high enough to allow cleavage of the protease recognition site by the protease but low enough to avoid interference with the analyte binding.

43. The detection reagent of claim 39 comprising a solid substrate and a protease, wherein the signaling moiety comprises an enzyme and the detection reagent further comprises a substrate for said enzyme that upon conversion by the enzyme leads to generation of a detectable signal, wherein said substrate is immobilized on a solid substrate such that the enzyme can only affect conversion once the enzyme is released from the immobilized peptide biosensor by cleavage of the protease recognition site by the protease.

44. Method of detecting the presence and/or amount of an analyte in a sample, comprising:
(i) contacting the detection reagent of claim 39 with a sample suspected of containing the analyte under conditions that allow binding of the analyte by the detection reagent; and
(ii) detecting the presence and/or amount of the analyte in said sample by measuring the signal of the signaling moiety.

45. Method of screening for compounds that modulate the binding of an analyte to a binding partner of the analyte, comprising
(i) contacting the detection reagent of claim 39 comprising a peptide biosensor and a protease with the analyte and a candidate compound under conditions that allow binding of the analyte by the detection reagent, wherein the analyte binding partner is the analyte binding site or the analyte binding molecule, optionally the first (poly)peptide capable of binding the analyte; and
(ii) measuring the signal of the signaling moiety, wherein a change in the signal compared to a reference not containing the candidate compound indicates modulation of the binding of the analyte and the analyte binding site or analyte binding molecule by the candidate compound.

46. The method of claim 45, wherein the compounds inhibit the binding of an analyte to a binding partner of the analyte.


SEQUENCE LISTING (23 sequences)

SEQ ID NO: 1 - protein, 5 aa, Artificial
  Enterokinase protease recognition site
DDDDR

SEQ ID NO: 2 - protein, 9 aa, Artificial
  Influenza virus hemagglutinin epitope
YPYDVPDYA

SEQ ID NO: 3 - protein, 12 aa, Artificial
  p53-derived binding site for mdm2
TSFAEYWNLLSP

SEQ ID NO: 4 - protein, 286 aa, Salmonella typhimurium [sequence omitted]

SEQ ID NO: 5 - protein, 1743 aa, Clostridium difficile
MKQNKLLQRGAYFNDKNILIDDFDKRYNDYDFVEFFTGISNSTFGLKSDGNLYACGDNTGFQLGLGKDSSERRMFSKVKIDNVKYVSCGSKHSVAVTKDGFAYGAGTSNVGQLGVIESTVYYEFTKLPIDDVKTVACGYDFTFVLKNDGTLYSAGLNSSGQLGLGDTNNRVTFTKVNIDSVKDVVTYNQSVFIIKMDGTAHACGLNSNGQLGINSTLNKSVFNKIEGMDNVKQIACGSSHTILIKNDGTMYTTGSNGYGQLGTGNNNNSIVFTLSSINNVKYASCGNNHTMILKYDNTLFSTGQNNYGQLANANKDVASRNTFVKVNVENIKDIKCGSQFNFLINGSKEIFVSGCNLAGQLGSFFHTTFLYEFSKVQSSNLDNYSGLLVNDDYLYVTKDNSEFLNVKLSDNFQDYKKIELTDNNMFIVMNDGTLYACGLNNYGQLGLGDTVNRSVMTKVDIDNVLDIKGNGNSTFVLKNNGTLYSCGYNSSGILGLKDNTNRNIFTKIEIENIKEFCVESNYIVALNHSKELYGWGNQSYIVYGDNRNYPYKDTRVSNVEKIATWSDTLYILDSTGATKTIGYSYNGSGGYPAPSSSSTYREGGYINKNTSYRTLEFYNTSKTKLVNLFAFYNGCVFVDENGLAYCIGENNINFRGGSTTNENNSLRFINNSGVYYTNTDGTDYTCYQWTYKLIRCSIFDSPQNIIGNSKNILYLSKNNSTFKCTGNCITYGINSQNWYSYFSDSSNGAIALGNEFILKNYSGECLLKGYGKATNGEFGNSTNISSISNYDTGLKDIKDIIVKNNTVVVVDKNNNIYVTGANQFNKLGIGEYNNQPIRKFTNITEQSNSFIFMDDIKEITTSRNTMFIVKNDGTAYATGNNSSGQLGLGDTINRNKFTQINLDNIKKISTSIDGNTTFAIRNDGTLYSTGLNTKGQLGLGDIVNRNTFTKVNIQNVRDVVLGTTHSHAIKDDNTLYSCGENTHGQLGLGSESNHPDVLTFTVNNITNVRDVYCSDTTTFIVKDTNIAYCCGYNNNSQLGMGNTTDQYSFIKCMENVKEVIPNEINTYIITIYNTAYSTGLNTDYCLGLNSNSNQSSFSEIPISNVVKVAPNRNNAVLLLTSEGDVYTAGKCSNGSGTGSETPEKIKKIASKAKDIGMNYRCGHYVSDNGDLYGTGFNDCGQLGVGNVTKRDTFIKTNTRVKKILPLEYANIAIKDTNDIYICGLNNYGQLGVGNRYDSRNNDNRIFNYKHMNFVMGDLTSIKNRHNFILLNNKIVIPTTKDIDYGLVLGNLYKGDLYTELPYEDIKEVSISKTHIIILLNDGTMYGCGTNYHGELLQDLSINQVDEFVQINVSDVKHVSCGDNFTYFIKSDDSLWSIGKNSEYQLGIGHNNPVTELQRITTISSCKEVHCGKNYTLVVTTSNELFVQGYNDKGALGLGSDSENTIIKFFTKALTDIREIKSYGSDHILVLKNDNSVWVTGKNRDVYKIEQPVEFLKEFTIVPISEDVNTVKDVLATDNTLYIISEVGTTNAAIEITEKSISSIKIKIQDPNKDISRIEMLINGESVKSVSDLITEKISFEVPPDKIKIGENKILFRAYCKGDDLYASLFIFKESTGNSIIKDSYVMIGNRMYKVVNTTSNEQDITITLDRGLEEDLNLGDPIYQLINKTKVQVKINKSDLFKDMKLVEIKKSDSSYQEIYELEEANIKSAQPKIIVEKGDKWTAIKRPSMIFRYDAENNEPQA

SEQ ID NO: 6 - protein, 24 aa, Artificial
  Artificial peptide, E at position 1 side chain modified with EDANS, K at position 10 side chain modified with Dabcyl
ESGDDDDRGKTSFAEYWNLLSPGS

SEQ ID NO: 7 - protein, 11 aa, Artificial
  p53 wt peptide
ETFSDLWKLLS

SEQ ID NO: 8 - protein, 11 aa, Artificial
  mutated p53 peptide
ETASDLAKLAP

SEQ ID NO: 9 - DNA, 45 bp, Artificial
  Oligonucleotide primer
aacatcgtacggatagccacggtcatcgtcatcgctaccgcccca

SEQ ID NO: 10 - DNA, 36 bp, Artificial
  Oligonucleotide primer
tatccgtacgatgttccggactacgccggaggtgtt

SEQ ID NO: 11 - DNA, 36 bp, Artificial
  Oligonucleotide primer
ctgaaaatacaggttttcgctaccgccccaatgttt

SEQ ID NO: 12 - DNA, 33 bp, Artificial
  Oligonucleotide primer
aacctgtattttcagtctggctatccgtacgat

SEQ ID NO: 13 - DNA, 33 bp, Artificial
  Oligonucleotide primer
acgatcgtcatcgtcaccactaccgccccaatg

SEQ ID NO: 14 - DNA, 42 bp, Artificial
  Oligonucleotide primer
gacgatgacgatcgtggtggtactagctttgcagaatattgg

SEQ ID NO: 15 - DNA, 42 bp, Artificial
  Oligonucleotide primer
gcagaatattggaacctgttgtctccgggatccgaagagatt

SEQ ID NO: 16 - DNA, 42 bp, Artificial
  Oligonucleotide primer
gacgatgacgatcgtggtggtaaaaagcgttatagccgtgat

SEQ ID NO: 17 - DNA, 42 bp, Artificial
  Oligonucleotide primer
tatagccgtgatcaactgttagcgctgggatccgaagagatt

SEQ ID NO: 18 - protein, 2057 aa, Artificial
  Peptide biosensor HA-enterokinase
MSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWKHWGGSDDDDRGYPYDVPDYAGGVMTMKQNKLLQRGAYFNDKNILIDDFDKRYNDYDFVEFFTGISNSTFGLKSDGNLYACGDNTGFQLGLGKDSSERRMFSKVKIDNVKYVSCGSKHSVAVTKDGFAYGAGTSNVGQLGVIESTVYYEFTKLPIDDVKTVACGYDFTFVLKNDGTLYSAGLNSSGQLGLGDTNNRVTFTKVNIDSVKDVVTYNQSVFIIKMDGTAHACGLNSNGQLGINSTLNKSVFNKIEGMDNVKQIACGSSHTILIKNDGTMYTTGSNGYGQLGTGNNNNSIVFTLSSINNVKYASCGNNHTMILKYDNTLFSTGQNNYGQLANANKDVASRNTFVKVNVENIKDIKCGSQFNFLINGSKEIFVSGCNLAGQLGSFFHTTFLYEFSKVQSSNLDNYSGLLVNDDYLYVTKDNSEFLNVKLSDNFQDYKKIELTDNNMFIVMNDGTLYACGLNNYGQLGLGDTVNRSVMTKVDIDNVLDIKGNGNSTFVLKNNGTLYSCGYNSSGILGLKDNTNRNIFTKIEIENIKEFCVESNYIVALNHSKELYGWGNQSYIVYGDNRNYPYKDTRVSNVEKIATWSDTLYILDSTGATKTIGYSYNGSGGYPAPSSSSTYREGGYINKNTSYRTLEFYNTSKTKLVNLFAFYNGCVFVDENGLAYCIGENNINFRGGSTTNENNSLRFINNSGVYYTNTDGTDYTCYQWTYKLIRCSIFDSPQNIIGNSKNILYLSKNNSTFKCTGNCITYGINSQNWYSYFSDSSNGAIALGNEFILKNYSGECLLKGYGKATNGEFGNSTNISSISNYDTGLKDIKDIIVKNNTVVVVDKNNNIYVTGANQFNKLGIGEYNNQPIRKFTNITEQSNSFIFMDDIKEITTSRNTMFIVKNDGTAYATGNNSSGQLGLGDTINRNKFTQINLDNIKKISTSIDGNTTFAIRNDGTLYSTGLNTKGQLGLGDIVNRNTFTKVNIQNVRDVVLGTTHSHAIKDDNTLYSCGENTHGQLGLGSESNHPDVLTFTVNNITNVRDVYCSDTTTFIVKDTNIAYCCGYNNNSQLGMGNTTDQYSFIKCMENVKEVIPNEINTYIITIYNTAYSTGLNTDYCLGLNSNSNQSSFSEIPISNVVKVAPNRNNAVLLLTSEGDVYTAGKCSNGSGTGSETPEKIKKIASKAKDIGMNYRCGHYVSDNGDLYGTGFNDCGQLGVGNVTKRDTFIKTNTRVKKILPLEYANIAIKDTNDIYICGLNNYGQLGVGNRYDSRNNDNRIFNYKHMNFVMGDLTSIKNRHNFILLNNKIVIPTTKDIDYGLVLGNLYKGDLYTELPYEDIKEVSISKTHIIILLNDGTMYGCGTNYHGELLQDLSINQVDEFVQINVSDVKHVSCGDNFTYFIKSDDSLWSIGKNSEYQLGIGHNNPVTELQRITTISSCKEVHCGKNYTLVVTTSNELFVQGYNDKGALGLGSDSENTIIKFFTKALTDIREIKSYGSDHILVLKNDNSVWVTGKNRDVYKIEQPVEFLKEFTIVPISEDVNTVKDVLATDNTLYIISEVGTTNAAIEITEKSISSIKIKIQDPNKDISRIEMLINGESVKSVSDLITEKISFEVPPDKIKIGENKILFRAYCKGDDLYASLFIFKESTGNSIIKDSYVMIGNRMYKVVNTTSNEQDITITLDRGLEEDLNLGDPIYQLINKTKVQVKINKSDLFKDMKLVEIKKSDSSYQEIYELEEANIKSAQPKIIVEKGDKWTAIKRPSMIFRYDAENNEPQAHA

SEQ ID NO: 19 - protein, 2070 aa, Artificial
  Peptide biosensor mdm2-enterokinase
MSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWKHWGGSGDDDDRGGTSFAEYWNLLSPGSEEIGLGAGVMTMKQNKLLQRGAYFNDKNILIDDFDKRYNDYDFVEFFTGISNSTFGLKSDGNLYACGDNTGFQLGLGKDSSERRMFSKVKIDNVKYVSCGSKHSVAVTKDGFAYGAGTSNVGQLGVIESTVYYEFTKLPIDDVKTVACGYDFTFVLKNDGTLYSAGLNSSGQLGLGDTNNRVTFTKVNIDSVKDVVTYNQSVFIIKMDGTAHACGLNSNGQLGINSTLNKSVFNKIEGMDNVKQIACGSSHTILIKNDGTMYTTGSNGYGQLGTGNNNNSIVFTLSSINNVKYASCGNNHTMILKYDNTLFSTGQNNYGQLANANKDVASRNTFVKVNVENIKDIKCGSQFNFLINGSKEIFVSGCNLAGQLGSFFHTTFLYEFSKVQSSNLDNYSGLLVNDDYLYVTKDNSEFLNVKLSDNFQDYKKIELTDNNMFIVMNDGTLYACGLNNYGQLGLGDTVNRSVMTKVDIDNVLDIKGNGNSTFVLKNNGTLYSCGYNSSGILGLKDNTNRNIFTKIEIENIKEFCVESNYIVALNHSKELYGWGNQSYIVYGDNRNYPYKDTRVSNVEKIATWSDTLYILDSTGATKTIGYSYNGSGGYPAPSSSSTYREGGYINKNTSYRTLEFYNTSKTKLVNLFAFYNGCVFVDENGLAYCIGENNINFRGGSTTNENNSLRFINNSGVYYTNTDGTDYTCYQWTYKLIRCSIFDSPQNIIGNSKNILYLSKNNSTFKCTGNCITYGINSQNWYSYFSDSSNGAIALGNEFILKNYSGECLLKGYGKATNGEFGNSTNISSISNYDTGLKDIKDIIVKNNTVVVVDKNNNIYVTGANQFNKLGIGEYNNQPIRKFTNITEQSNSFIFMDDIKEITTSRNTMFIVKNDGTAYATGNNSSGQLGLGDTINRNKFTQINLDNIKKISTSIDGNTTFAIRNDGTLYSTGLNTKGQLGLGDIVNRNTFTKVNIQNVRDVVLGTTHSHAIKDDNTLYSCGENTHGQLGLGSESNHPDVLTFTVNNITNVRDVYCSDTTTFIVKDTNIAYCCGYNNNSQLGMGNTTDQYSFIKCMENVKEVIPNEINTYIITIYNTAYSTGLNTDYCLGLNSNSNQSSFSEIPISNVVKVAPNRNNAVLLLTSEGDVYTAGKCSNGSGTGSETPEKIKKIASKAKDIGMNYRCGHYVSDNGDLYGTGFNDCGQLGVGNVTKRDTFIKTNTRVKKILPLEYANIAIKDTNDIYICGLNNYGQLGVGNRYDSRNNDNRIFNYKHMNFVMGDLTSIKNRHNFILLNNKIVIPTTKDIDYGLVLGNLYKGDLYTELPYEDIKEVSISKTHIIILLNDGTMYGCGTNYHGELLQDLSINQVDEFVQINVSDVKHVSCGDNFTYFIKSDDSLWSIGKNSEYQLGIGHNNPVTELQRITTISSCKEVHCGKNYTLVVTTSNELFVQGYNDKGALGLGSDSENTIIKFFTKALTDIREIKSYGSDHILVLKNDNSVWVTGKNRDVYKIEQPVEFLKEFTIVPISEDVNTVKDVLATDNTLYIISEVGTTNAAIEITEKSISSIKIKIQDPNKDISRIEMLINGESVKSVSDLITEKISFEVPPDKIKIGENKILFRAYCKGDDLYASLFIFKESTGNSIIKDSYVMIGNRMYKVVNTTSNEQDITITLDRGLEEDLNLGDPIYQLINKTKVQVKINKSDLFKDMKLVEIKKSDSSYQEIYELEEANIKSAQPKIIVEKGDKWTAIKRPSMIFRYDAENNEPQAHA

SEQ ID NO: 20 - protein, 2059 aa, Artificial
  Peptide biosensor HA-TEV protease
MSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWKHWGGSENLYFQSGYPYDVPDYAAGVMTMKQNKLLQRGAYFNDKNILIDDFDKRYNDYDFVEFFTGISNSTFGLKSDGNLYACGDNTGFQLGLGKDSSERRMFSKVKIDNVKYVSCGSKHSVAVTKDGFAYGAGTSNVGQLGVIESTVYYEFTKLPIDDVKTVACGYDFTFVLKNDGTLYSAGLNSSGQLGLGDTNNRVTFTKVNIDSVKDVVTYNQSVFIIKMDGTAHACGLNSNGQLGINSTLNKSVFNKIEGMDNVKQIACGSSHTILIKNDGTMYTTGSNGYGQLGTGNNNNSIVFTLSSINNVKYASCGNNHTMILKYDNTLFSTGQNNYGQLANANKDVASRNTFVKVNVENIKDIKCGSQFNFLINGSKEIFVSGCNLAGQLGSFFHTTFLYEFSKVQSSNLDNYSGLLVNDDYLYVTKDNSEFLNVKLSDNFQDYKKIELTDNNMFIVMNDGTLYACGLNNYGQLGLGDTVNRSVMTKVDIDNVLDIKGNGNSTFVLKNNGTLYSCGYNSSGILGLKDNTNRNIFTKIEIENIKEFCVESNYIVALNHSKELYGWGNQSYIVYGDNRNYPYKDTRVSNVEKIATWSDTLYILDSTGATKTIGYSYNGSGGYPAPSSSSTYREGGYINKNTSYRTLEFYNTSKTKLVNLFAFYNGCVFVDENGLAYCIGENNINFRGGSTTNENNSLRFINNSGVYYTNTDGTDYTCYQWTYKLIRCSIFDSPQNIIGNSKNILYLSKNNSTFKCTGNCITYGINSQNWYSYFSDSSNGAIALGNEFILKNYSGECLLKGYGKATNGEFGNSTNISSISNYDTGLKDIKDIIVKNNTVVVVDKNNNIYVTGANQFNKLGIGEYNNQPIRKFTNITEQSNSFIFMDDIKEITTSRNTMFIVKNDGTAYATGNNSSGQLGLGDTINRNKFTQINLDNIKKISTSIDGNTTFAIRNDGTLYSTGLNTKGQLGLGDIVNRNTFTKVNIQNVRDVVLGTTHSHAIKDDNTLYSCGENTHGQLGLGSESNHPDVLTFTVNNITNVRDVYCSDTTTFIVKDTNIAYCCGYNNNSQLGMGNTTDQYSFIKCMENVKEVIPNEINTYIITIYNTAYSTGLNTDYCLGLNSNSNQSSFSEIPISNVVKVAPNRNNAVLLLTSEGDVYTAGKCSNGSGTGSETPEKIKKIASKAKDIGMNYRCGHYVSDNGDLYGTGFNDCGQLGVGNVTKRDTFIKTNTRVKKILPLEYANIAIKDTNDIYICGLNNYGQLGVGNRYDSRNNDNRIFNYKHMNFVMGDLTSIKNRHNFILLNNKIVIPTTKDIDYGLVLGNLYKGDLYTELPYEDIKEVSISKTHIIILLNDGTMYGCGTNYHGELLQDLSINQVDEFVQINVSDVKHVSCGDNFTYFIKSDDSLWSIGKNSEYQLGIGHNNPVTELQRITTISSCKEVHCGKNYTLVVTTSNELFVQGYNDKGALGLGSDSENTIIKFFTKALTDIREIKSYGSDHILVLKNDNSVWVTGKNRDVYKIEQPVEFLKEFTIVPISEDVNTVKDVLATDNTLYIISEVGTTNAAIEITEKSISSIKIKIQDPNKDISRIEMLINGESVKSVSDLITEKISFEVPPDKIKIGENKILFRAYCKGDDLYASLFIFKESTGNSIIKDSYVMIGNRMYKVVNTTSNEQDITITLDRGLEEDLNLGDPIYQLINKTKVQVKINKSDLFKDMKLVEIKKSDSSYQEIYELEEANIKSAQPKIIVEKGDKWTAIKRPSMIFRYDAENNEPQAHA

SEQ ID NO: 21 - protein, 2069 aa, Artificial
  Peptide biosensor eiF4E-enterokinase
MSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWKHWGGSGDDDDRGKKRYSRDQLLALGSEEIGLGAGVMTMKQNKLLQRGAYFNDKNILIDDFDKRYNDYDFVEFFTGISNSTFGLKSDGNLYACGDNTGFQLGLGKDSSERRMFSKVKIDNVKYVSCGSKHSVAVTKDGFAYGAGTSNVGQLGVIESTVYYEFTKLPIDDVKTVACGYDFTFVLKNDGTLYSAGLNSSGQLGLGDTNNRVTFTKVNIDSVKDVVTYNQSVFIIKMDGTAHACGLNSNGQLGINSTLNKSVFNKIEGMDNVKQIACGSSHTILIKNDGTMYTTGSNGYGQLGTGNNNNSIVFTLSSINNVKYASCGNNHTMILKYDNTLFSTGQNNYGQLANANKDVASRNTFVKVNVENIKDIKCGSQFNFLINGSKEIFVSGCNLAGQLGSFFHTTFLYEFSKVQSSNLDNYSGLLVNDDYLYVTKDNSEFLNVKLSDNFQDYKKIELTDNNMFIVMNDGTLYACGLNNYGQLGLGDTVNRSVMTKVDIDNVLDIKGNGNSTFVLKNNGTLYSCGYNSSGILGLKDNTNRNIFTKIEIENIKEFCVESNYIVALNHSKELYGWGNQSYIVYGDNRNYPYKDTRVSNVEKIATWSDTLYILDSTGATKTIGYSYNGSGGYPAPSSSSTYREGGYINKNTSYRTLEFYNTSKTKLVNLFAFYNGCVFVDENGLAYCIGENNINFRGGSTTNENNSLRFINNSGVYYTNTDGTDYTCYQWTYKLIRCSIFDSPQNIIGNSKNILYLSKNNSTFKCTGNCITYGINSQNWYSYFSDSSNGAIALGNEFILKNYSGECLLKGYGKATNGEFGNSTNISSISNYDTGLKDIKDIIVKNNTVVVVDKNNNIYVTGANQFNKLGIGEYNNQPIRKFTNITEQSNSFIFMDDIKEITTSRNTMFIVKNDGTAYATGNNSSGQLGLGDTINRNKFTQINLDNIKKISTSIDGNTTFAIRNDGTLYSTGLNTKGQLGLGDIVNRNTFTKVNIQNVRDVVLGTTHSHAIKDDNTLYSCGENTHGQLGLGSESNHPDVLTFTVNNITNVRDVYCSDTTTFIVKDTNIAYCCGYNNNSQLGMGNTTDQYSFIKCMENVKEVIPNEINTYIITIYNTAYSTGLNTDYCLGLNSNSNQSSFSEIPISNVVKVAPNRNNAVLLLTSEGDVYTAGKCSNGSGTGSETPEKIKKIASKAKDIGMNYRCGHYVSDNGDLYGTGFNDCGQLGVGNVTKRDTFIKTNTRVKKILPLEYANIAIKDTNDIYICGLNNYGQLGVGNRYDSRNNDNRIFNYKHMNFVMGDLTSIKNRHNFILLNNKIVIPTTKDIDYGLVLGNLYKGDLYTELPYEDIKEVSISKTHIIILLNDGTMYGCGTNYHGELLQDLSINQVDEFVQINVSDVKHVSCGDNFTYFIKSDDSLWSIGKNSEYQLGIGHNNPVTELQRITTISSCKEVHCGKNYTLVVTTSNELFVQGYNDKGALGLGSDSENTIIKFFTKALTDIREIKSYGSDHILVLKNDNSVWVTGKNRDVYKIEQPVEFLKEFTIVPISEDVNTVKDVLATDNTLYIISEVGTTNAAIEITEKSISSIKIKIQDPNKDISRIEMLINGESVKSVSDLITEKISFEVPPDKIKIGENKILFRAYCKGDDLYASLFIFKESTGNSIIKDSYVMIGNRMYKVVNTTSNEQDITITLDRGLEEDLNLGDPIYQLINKTKVQVKINKSDLFKDMKLVEIKKSDSSYQEIYELEEANIKSAQPKIIVEKGDKWTAIKRPSMIFRYDAENNEPQAHA

SEQ ID NO: 22 - DNA, 36 bp, Artificial; identical to SEQ ID NO: 10
  Oligonucleotide primer

SEQ ID NO: 23 - DNA, 45 bp, Artificial
  Oligonucleotide primer
aacatcgtacggataaccacgcggaaccagaccgctaccgcccca